(19) United States
(12) Patent Application Publication
(10) Pub. No.: US 2024/0023809 A1
(43) Pub. Date: Jan. 25, 2024

(54) Systems and Methods for Detection of Musculoskeletal Anomalies

(71) Applicant: The Board of Trustees of the Leland Stanford Junior University, Stanford, CA (US)

(72) Inventors: Malcolm R. DeBaun, Menlo Park, CA (US); Michael J. Gardner, Menlo Park, CA (US)

(73) Assignee: The Board of Trustees of the Leland Stanford Junior University, Stanford, CA (US)

(21) Appl. No.: 17/759,919

(22) PCT Filed: Feb. 1, 2021
(86) PCT No.: PCT/US21/16088

§ 371 Date: Aug. 1, 2022

Related U.S. Application Data
(60) Provisional application No. 63/130,291, filed on Dec. 23, 2020.
(60) Provisional application No. 62/968,884, filed on Jan. 31, 2020.

(51) Int. Cl. A61B 5/00; A61B 5/107; G01S 17/89; G06T 15/08; G06T 7/00

(52) U.S. Cl. CPC A61B 5/0064; A61B 5/0013; A61B 5/0046; A61B 5/1071; A61B 5/4561; A61B 5/4576; A61B 5/4848; G01S 17/89; G06T 15/08; G06T 7/0012; A61B 2560/0431; A61B 2576/02

(57) ABSTRACT

Systems and methods for the detection of musculoskeletal anomalies are disclosed. Various embodiments are directed to methods to detect and treat anomalies, including fracture (e.g. clavicle), deformity (e.g. scoliosis), and other anomalies. Various embodiments utilize structured white light scanners, while additional embodiments utilize LiDAR to generate 3-dimensional (3D) topographic scans. Various embodiments obtain these scans via a mobile device, such as a mobile phone or tablet. Further embodiments utilize machine learning models to analyze the 3D scans to identify an anomaly and/or a treatment for such anomaly and/or monitor change of that condition over time.

### CROSS REFERENCE TO RELATED APPLICATIONS

This application claims priority to U.S. Provisional Application Ser. No. 63/130,291, entitled “Systems and Methods for Detection of Musculoskeletal Anomalies” to DeBaun et al., filed Dec. 23, 2020 and U.S. Provisional Application Ser. No. 62/968,884, entitled “Systems and Methods for Fracture Detection” to DeBaun et al., filed Jan. 31, 2020; the disclosures of which are hereby incorporated by reference in their entireties.

### STATEMENT REGARDING FEDERALLY SPONSORED RESEARCH OR DEVELOPMENT

This invention was made with Government support under TR003142 awarded by the National Institutes of Health. The Government has certain rights in this invention.

### FIELD OF THE INVENTION

The present invention is directed to systems for performing musculoskeletal analyses. More particularly, the present invention is directed to systems incorporating three-dimensional (3D) scans and machine learning to identify musculoskeletal abnormalities and conditions, such as fracture, deformity, asymmetry, center of gravity or rotation, and joint range of motion.

### BACKGROUND OF THE INVENTION

Musculoskeletal problems are significant problems in human populations, including bone fractures, scoliosis, and other deformities or anomalies. Such problems are conventionally observed using X-ray imaging. X-rays provide high contrast images of bones against other soft tissues. However, X-rays are a form of relatively high-energy electromagnetic radiation which can adversely affect organic tissue. While modern X-ray imaging devices are designed to use as little radiation as possible, repeated exposure can still cause harm to patients. For conditions such as a clavicle fracture, doctors generally rely on objective radiographic criteria not only in order to diagnose and suggest treatment, but to follow up on recovery progression over time, thereby increasing their radiation damage burden. Additionally, much the equipment for obtaining these measurements are not mobile or easily dispatched into a field setting. Furthermore, X-rays are insufficient methods of detecting or monitoring many musculoskeletal problems, because they provide only a 2-dimensional representation of bony anatomy (which fails to represent 3-dimensional nature of a deformity after a fracture) and fail to capture soft tissues (which can be critical for medical diagnosis).

While some issues, like scoliosis, are diagnosed in the field, such screening techniques are rudimentary and inaccurate, leading to inappropriate referrals and increased patient anxiety. Further confirmation and monitoring of scoliosis require additional medical imaging, such as X-ray, which has many of the problems, previously described.

### SUMMARY OF THE INVENTION

Systems and methods for detection of musculoskeletal anomalies are provided. In one embodiment, a three dimensional diagnostic system includes a three dimensional scanning device capable of obtaining a three dimensional scan of a human body without emitting ionizing or other damaging radiation and a computing device in communication with the three dimensional scanning device and capable of generating a mesh from a three dimensional scan and analyzing said mesh to identify a musculoskeletal anomaly.

In a further embodiment, the three dimensional scanning device is a white light scanning camera or a LiDAR-enabled camera.

In another embodiment, the computing device is a mobile device.

In a still further embodiment, the mobile device is selected from a mobile phone, a tablet, a laptop computer, or a notebook computer.

In still another embodiment, the computing device is capable of transmitting data over a network.

In a yet further embodiment, the system further includes a remote server connected to the computing device via a network.

In yet another embodiment, a method for detecting and monitoring scoliosis includes obtaining a 3D topographic scan of a subject's body using a 3D topographic imaging device, analyzing the 3D topographic scan by identifying a plurality of key feature points on the regions of the 3D topographic scan reflecting the back of the subject, measuring a distance or angle between at least a first key feature point and a second key feature point in the plurality of key feature points, identifying scoliosis based on the distances, angles, and volumetric relationships quantified in upright and bending poses, classifying the scoliosis as in need of orthopaedic referral or not in need of orthopaedic referral, classifying the scoliosis as operative, eligible for casting and/or bracing or not in need of intervention, and treating the subject based on the classification of the scoliosis.

In a further embodiment again, the plurality of key features are selected from the group consisting of: the midsternal notch, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

In another embodiment again, the treating step includes a surgical operation, other non-surgical intervention, or physical therapy.

In a further additional embodiment, the method further includes obtaining a second 3D topographic scan of the subject's body post-treatment, identifying a second plurality of key feature points in the second 3D topographic scan using a fracture detector, measuring a distance, angle, or volumetric change between at least a first key feature point and a second key feature point in the second plurality of key feature points using the fracture detector, calculating the difference in the measured distance, angles or volumetric change, and tracking the subject's recovery based on the calculated differences in distances, angles or volumetric measurements of interest.

In another additional embodiment, the plurality of key features are selected from the group consisting of: the midsternal notch, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

In a still yet further embodiment, the 3D topographic scan is accomplished using a structured light scanner or LiDAR-enabled camera.

In still yet another embodiment, the 3D topographic scan is accomplished using a mobile device.

In a still further embodiment again, the mobile device is selected from a mobile phone or tablet.

In still another embodiment again, a method for detecting and treating clavicle fractures includes obtaining a 3D topographic scan of a subject's body using a 3D topographic imaging device, identifying a plurality of key feature points on the regions of the 3D topographic scan reflecting the shoulders and back of the subject, measuring a distance between at least a first key feature point and a second key feature point in the plurality of key feature points, identifying a clavicle fracture based on the distance, classifying the clavicle fracture as operative or non-operative, and treating the subject based on the classification of the clavicle fracture.

In a still further additional embodiment, the plurality of key features are selected from the group consisting of: the midsternal notch, the acromial process, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

In still another additional embodiment, the treating step includes a surgical operation.

In a yet further embodiment again, the method further includes obtaining a second 3D topographic scan of the subject's body post-operatively, identifying a second plurality of key feature points in the second 3D topographic scan using a fracture detector, measuring a distance between at least a first key feature point and a second key feature point in the second plurality of key feature points using the fracture detector, calculating the difference in the measured distances, calculating volumetric relationships within 3D scans, and tracking the subject's recovery based on the calculated differences.

In yet another embodiment again, the plurality of key features are selected from the group consisting of: the midsternal notch, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

In a yet further additional embodiment, the 3D topographic scan is accomplished using a structured light scanner or LiDAR-enabled camera.

In yet another additional embodiment, the 3D topographic scan is accomplished using a mobile device.

In a further additional embodiment again, the mobile device is selected from a mobile phone or tablet.

In another additional embodiment again, a method for detecting musculoskeletal anomalies includes obtaining a 3D topographic scan of a subject's body using a 3D topographic imaging device, performing range of motion, center of gravity, asymmetry, or posture analysis on the 3D topographic scan by bisecting the scan with one or more lines and measuring a key feature along the one or more lines, and identifying a musculoskeletal anomaly based on the distance.

In a still yet further embodiment again, the 3D topographic scan is accomplished using a structured light scanner or LiDAR-enabled camera.

In still yet another embodiment again, the 3D topographic scan is accomplished using a mobile device.

In a still yet further additional embodiment, the mobile device is selected from a mobile phone or tablet.

In still yet another additional embodiment, the musculoskeletal anomaly is selected from scoliosis, back pain, neck pain, joint pain, sarcopenia, arthritis, osteoporosis, bone and soft tissue injury.

In a yet further additional embodiment again, obtaining the 3D topographic scan is accomplished by converting one or more two-dimensional images into a 3D representation of the subject's body.

### BRIEF DESCRIPTION OF THE DRAWINGS

These and other features and advantages of the present invention will be better understood by reference to the following detailed description when considered in conjunction with the accompanying drawings where:

FIG. 1A illustrates a method for detecting and treating musculoskeletal anomalies in accordance with various embodiments of the invention.

FIGS. 1B-1H illustrates exemplary measurements for analyzing specific features in accordance with various embodiments of the invention.

FIG. 2 illustrates an exemplary system for detecting and treating musculoskeletal anomalies in accordance with various embodiments of the invention.

FIG. 3 illustrates exemplary results of scores for injured and non-injured clavicles in accordance with various embodiments of the invention.

FIG. 4 illustrates exemplary results of a shoulder deformity identified in surgical and non-surgical patients over time in accordance with various embodiments of the invention.

FIG. 5 illustrates hand measurements for shoulder deformities for surgical and non-surgical patients in accordance with various embodiments of the invention.

FIG. 6A-6I illustrate exemplary comparisons of 3D scans, cobb angle, and a scoliometer in accordance with various embodiments of the invention.

FIGS. 7A-7L illustrate exemplary correlations between Patient Reported Outcome Measurements (PROMs) in radiography, 3D scans, and scolimetry in accordance with various embodiments of the invention.

### DETAILED DESCRIPTION

Turning now to the diagrams and figures, embodiments of the invention are generally directed to systems and methods to detect musculoskeletal anomalies. Systems and methods described herein can use visible spectrum light to obtain a three dimensional (3D) topographic scan of a patient and use the scan to detect musculoskeletal conditions such as, but not limited to, fractures, and/or any other musculoskeletal injury or anomaly as appropriate to the requirements of specific applications of embodiments of the invention. Specific addressable conditions include but are not limited to, scoliosis, back pain, neck pain, joint pain, sarcopenia, arthritis, osteoporosis, bone and soft tissue injury, flexibility, mobility, muscle strength, imbalances, posture analysis, body, bone, fat, muscle mass, metabolic rate, circumference and volume of various body parts. In many embodiments, the 3D scan describes the surface of the patient's body, and contains no internal information (e.g., a mesh).

By measuring particular key feature points, an accurate estimation can be made of whether or not an injury, deformity, or degeneration over time has occurred. In numerous embodiments, as supported by empirical studies, measurement of the same key feature points as performed by a human render less precise results. Many embodiments are deployed as portable devices, including as attached to or as part of a mobile phone or tablet to allow systems to be deployed outside of clinics, hospitals, or other medical facilities.

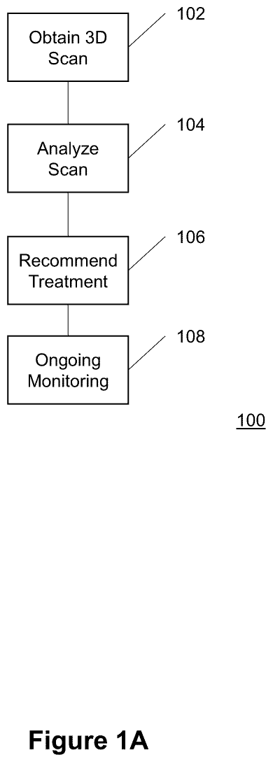

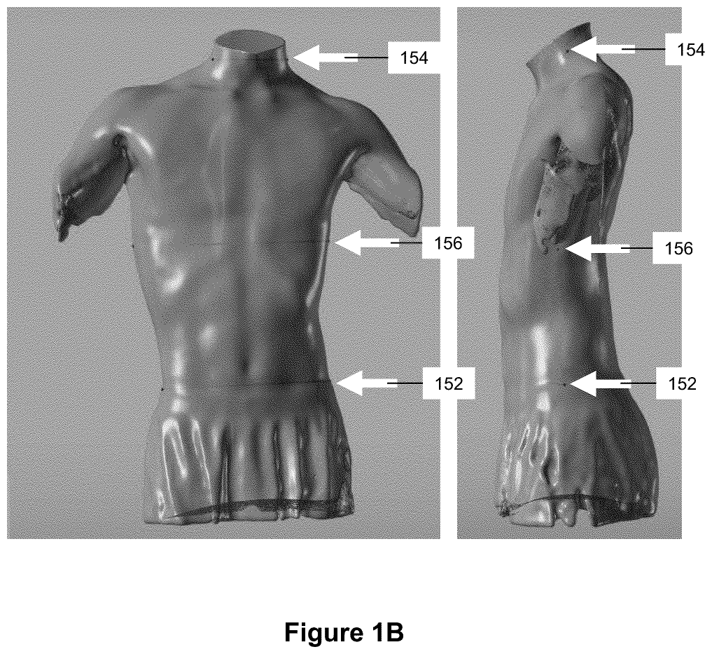

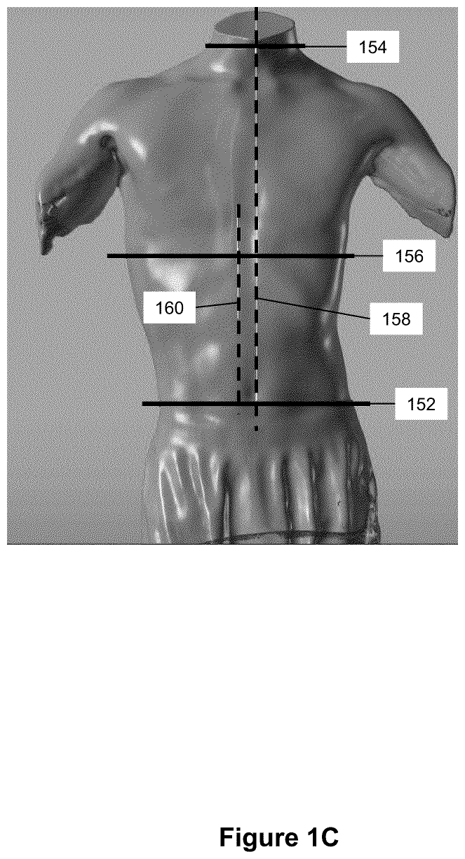

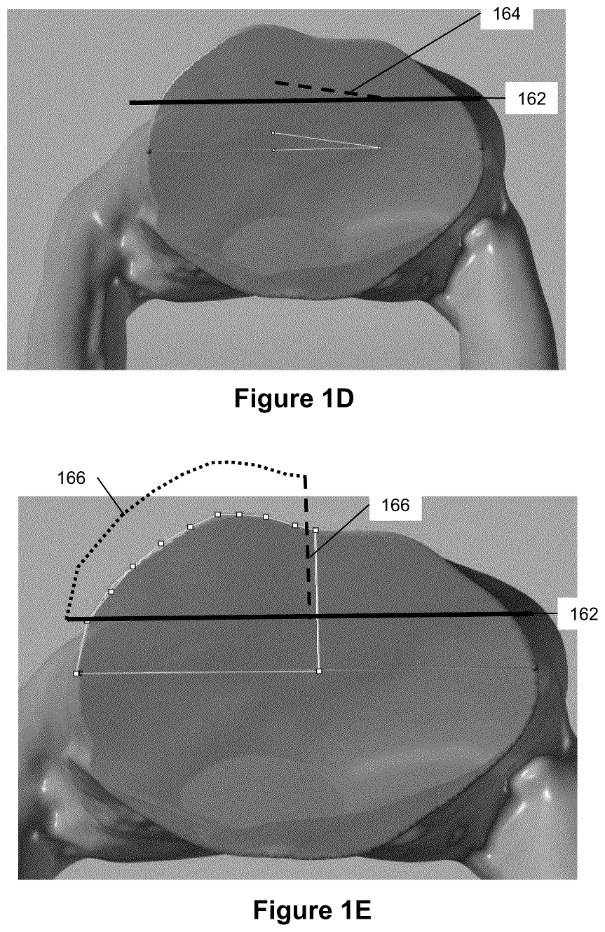

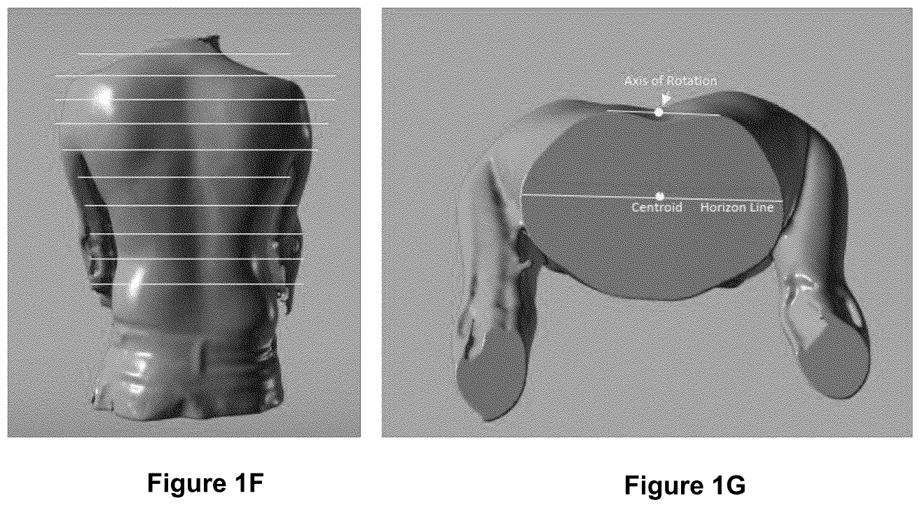

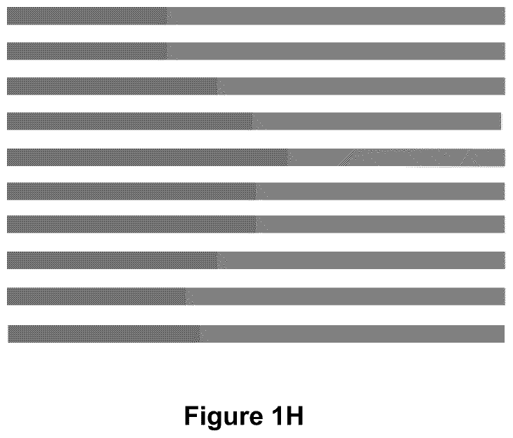

Turning to FIG. 1A, an exemplary embodiment describing a method 100 to treat an individual for musculoskeletal anomaly is illustrated. At 102, many embodiments obtain a 3D scan of an individual in a predefined pose dependent on the suspected musculoskeletal deformity. In many embodiments, a three-dimensional scanner, such as, but not limited to, a structured light scanner, LiDAR-enabled camera, including LiDAR-enabled cameras within newer mobile device and tablet models, or other light-based imaging system, is used to capture the 3-dimensional shape of, and/or quantify asymmetry of the human body. In numerous embodiments, data can but does not require encryption and automatically handled in a HIPAA compliant manner unless specified for medical legal compliance. In various embodiments, the 3D scan creates a mesh (e.g., solid file) of the body based on the 3D scan. In some embodiments, holes in the mesh are patched. In many embodiments, multiple 3D scans are obtained of an individual. For example, anterior and posterior views.

Additionally, some embodiments obtain 3D scans of an individual in different positions, such as standing, bending, sitting, and/or any other position relevant for a particular purpose. Some embodiments obtain 3D scans taken of an individual in a bent position (e.g., Adam's Forward Bend position) to maximally expose spinal curvature. In certain embodiments, 3D scans are obtained with one or both arms, one or both legs, and/or the head/neck in a specific position that allows for measurements of particular features dependent upon specific motions or specific positions of one or more appendages. Further embodiments obtain 3D video scans of an individual, in that the 3D scan is obtained as a continuous series of images over time. Certain embodiments construct 3D images of an individual based on one or more 2D images or a video that can be converted into a single 3D representation, via stitching, reconstruction, or other method of constructing a 3D representation from one or more 2D images.

At 104 of various embodiments, 3D representations are analyzed to identify a musculoskeletal anomaly. In numerous embodiments, systems and methods described herein can identify shoulder and spinal deformities such as clavicle fractures, scoliosis, and other deformities, diseases, or anomalies. In some embodiments, analysis is accomplished by communicating scans across a network and/or via the cloud to a central server for processing, while some embodiments analyze the 3D scans locally.

In many embodiments, the 3D representation is analyzed by demarcating various lines or representations and identifying ratios, angles, torsions, rotations, or other differences between these lines.

In various embodiments, a three-dimensional plane is demarcated from the center of the midsternal notch/umbilicus and the C7 spinous process/intergluteal cleft to divide the body into halves. Palpable anatomic landmarks can be utilized as key feature points such as, but not limited to: the midsternal notch, the superior/anterior aspect of the acromioclavicular joint, the posterior/lateral border of the acromion, the C7 spinous process, the acromial process, the inferior angle of the scapula, the nipples, the olecranon process, the anterior superior iliac spine, greater trochanter, patella, ankle malleoli, and digits. Many embodiments analyze distances between these landmarks, angles formed between these landmarks, and volumetric differences between areas of interest as well as between these landmarks and the ground to demonstrate the magnitude of topographical asymmetry.

In many embodiments, volumetric asymmetry is calculated through targeted volumetric comparison of the two halves of the body utilizing aforementioned anatomic landmarks. Asymmetric dynamic motion of the limb can also be calculated by comparing the change in relationships between the aforementioned landmarks through a range of motion. Output can be represented as absolute and relative asymmetry compared to the respective contralateral body part of interest or with respect to prior measurements demonstrating change over time.

FIG. 1B illustrates an exemplary method of analyzing 3D representations in accordance with some embodiments. Some embodiments draw three axial lines on the body, with a first axial line 152 located at the hip or pelvis, a second axial line 154 located at the neck, and a third axial line 156 located at an area of the trunk with largest deformity. In such embodiments, each line passes from the left midsagittal line to right midsagittal line. In some embodiments, the 3D lines are drawn parallel to the ground which is intrinsically to the 3D representation. In many embodiments, the area of largest deformity along the inferior/superior axis is determined by taking axial cuts throughout the torso and measuring the difference in area between left and right sagittal hemispheres within the two dimensional (2D) contour created by the axial cut. The location where this difference is at a maximum is annotated, and the third axial line 156 is drawn from left mid-sagittal line to right mid-sagittal line is drawn to cross through this point.

Turning to FIG. 1C, further embodiments determine coronal balance 158 and trunk shift 160 of the individual. In many embodiments, coronal balance is defined as a shift in a midpoint of the first axial line 152 and a midpoint of the second axial line 154, while the trunk shift is defined as a shift in a midpoint of the first axial line 152 and a midpoint of the third axial line 156.

To measure an angle of trunk rotation, such as for scoliosis, various embodiments utilize scans from a bent position (e.g., Adam's Forward Bend position). In such embodiments, slices are obtained from the 3D representations perpendicular to the mid-sagittal line along the torso and the points of largest deformity are identified in the lower back (lumbar spine) and upper back (thoracic spine). As illustrated in FIG. 1D, a horizon line 162 is determined by connecting a left midsagittal line with a right midsagittal line. The angle of trunk rotation 164 is the angle made by the posterior portion of the slice compared to the horizon line 162. FIG. 1E illustrates examples of displacement and circumferential distance measured from relative to a horizon line 162 in accordance with various embodiments. In many embodiments, displacement is calculated from the left midsagittal line to the projection 166 of the axis of rotation onto the horizon line and subsequently from the right midsagittal line to the projection of the axis of rotation onto the horizon line. Additionally, certain embodiments calculate circumferential distance 168 as a distance from the left midsagittal line to axis of rotation and right midsagittal line to axis of rotation.

Furth embodiments expand on these abilities by mapping spinal curvature from 3D representations or scans in any position, including at least one of standing, sitting, and bending. FIGS. 1F-1G illustrate on method in accordance with some embodiments to map spinal curvature. In particular, a plurality of axial slices 168 are made parallel to the ground. Within each slice 168, rotation is identified based on an angle created by the posterior portion of the torso and the horizon line 162. Further, displacement of the axis of rotation 170 in each slice 168 is measured from the mid-sagittal line is identified based on bilateral measurements 172, 172′ between the axis of rotation 170 and a centroid 174. With measurements obtained from each slice, 3D reconstructions can be reassembled for the spinal curvature. An exemplary displacement is illustrated in FIG. 1H that can be used to approximate spinal curvature. With such reconstructions, these embodiments can quantify left versus right asymmetry, deformity, and/or deformation, either statically or over time. Such methods facilitate detection and monitoring of other orthopedic conditions (osteoporosis, frailty, arthritis, back/neck pain, injury, and/or other malformations).

In various embodiments, systems and methods described herein can quantify static and dynamic asymmetry of the body to assist as a decision aid for medical decision making or personal wellness monitoring. For example, shoulder deformity after isolated clavicle fracture can be detected utilizing this methodology. In some embodiments, when diagnosing clavicle fracture, a structured-light scanner captures the three-dimensional shape of the shoulder girdle bilaterally. Non-traumatic musculoskeletal deformity, such as that in scoliosis, may also be captured utilizing similar methodology. In some embodiments, scoliotic deformity may be quantified by capturing the three-dimensional shape of the spine in upright and bending poses. Data captured from three-dimensional scans and then uploaded to a photogrammetric musculoskeletal software for analysis of three-dimensional measurements of anatomical relationships based upon specific landmarks.

In some embodiments, these palpable and visible anatomic landmarks are validated through academic clinical trials. For example, for a clavicle fracture, the specific landmarks include suprasternal notch, superior/anterior aspect of the acromioclavicular joint, posterior/lateral border of the acromion, inferior angle of the scapula, and C7 spinous process. Distance and the angles formed between these chosen landmarks are analyzed with the software to demonstrate the magnitude of topographical asymmetry compared to the injured and uninjured side. The injured and uninjured shoulder girdles are compared, with each patient to serve as their internal reference. The relative difference in the shoulder ptosis defined by these anatomic landmarks, specifically the distance from the midsternal notch to the acromial clavicular joint can identify displaced clavicle fractures that would benefit from operative management without the use of radiation. Further, the difference in distance and angles formed by the aforementioned landmarks is analyzed to monitor the restoration of anatomy or persistence of deformity to monitor the healing of their fracture without the use of radiation. Manual surface measurements of the landmarks are not as predictive due to a lack of sensitivity, thus validating the digital technology and methods. The restoration of anatomy or presence of persistent deformity after clavicle fracture as identified by described methodologies have predictive clinical relevance in terms of pain and return to function defined by objective outcome scores.

In an exemplary embodiment assessing scoliosis, upright and bending scans are used in conjunction to quantify three-dimensional scoliotic deformity. First, the location of the hip joints is estimated using the center of mass of each leg. The mid-point between the two estimated hip joints is then found to estimate the central sacral vertical line. The circumference of the torso is calculated transversely from cranial to caudal, and orthogonal line is drawn through the center of mass of the circumference on the transverse plane with the largest asymmetry. This line is projected onto a coronal plane and compared to the projection of the central sacral vertical line on the same coronal plane to calculate trunk shift. Next, a circumference is drawn transversely around the neck, and an orthogonal line is drawn through the center of mass of this circumference. The orthogonal line is projected onto the same coronal plane previously used in the trunk shift calculation, and is compared to the projection of the central sacral vertical line to determine coronal balance. Shoulder balance and clavicle angle are calculated using the anterior or posterior acromioclavicular joints, dependent on which is most prominent on a particular patient. In the case that these landmarks are not easily seen, the apex of each shoulder is compared to generate the same calculation. For calculation of angle of trunk rotation, splines are drawn from the estimated location of the hips to the center of the shoulder as determined from anterior to posterior. Lines are drawn from one side to another, and the angle of the back is compared to the coronal line created by the splines along the back. The largest angle in the lumbar spine is the lumbar angle of trunk rotation, and the largest angle in the thoracic spine is the thoracic angle of trunk rotation.

The aforementioned clinical scenarios, are just examples of utilization of these methods detects and monitor musculoskeletal abnormalities or conditions. Other clinical applications of similar methods include but are not limited to neck and back pain/injury, arthritis, osteoporosis, sarcopenia, soft tissue injury, laxity, and/or muscle atrophy/hypertrophy.

In many embodiments, the analysis is based on a machine learning model. In many embodiments, the machine learning model is trained from 3D scans of individuals, including individuals with new anomalies as well as individuals with categorized anomalies, such as clavicle fracture, scoliosis, a deformity, and/or any other anomaly. Further embodiments further include treatment prognostics or outlook, such as probable outcome from surgical intervention, physical therapy, sports medicine, pharmacological/pharmaceutical therapy (e.g., pain management), and/or other clinical treatment.

Returning to FIG. 1A, at 106, many embodiments generate recommendations for treatment specified to the type and severity of identified conditions. For example, in many embodiments, conditions can be classified as operative or non-operative, a particular surgical treatment can be recommended, and/or any other treatment regime can be selected based on identified conditions as appropriate to the requirements of specific applications of embodiments of the invention. In some embodiments, the prognostics are based on severity of any such anomaly, such that severe cases may recommend surgical intervention, while less severe cases may recommend less invasive intervention, such as monitoring, bracing/casting, and/or physical therapy.

At 106, embodiments may also generate recommendations for further evaluation by a specialist. For example, in scoliosis, a primary care provider or school nurse may obtain a three-dimensional scan and use the information obtained to determine whether referral to or discussion with an orthopaedic specialist is indicated.

Ongoing monitoring occurs at 108 of many embodiments. During ongoing monitoring, these embodiments follow up with a patient or individual via out-of-office surveys (e.g., patient-reported outcome measures, or PROM) or in-office examination, such as for freedom of movement, range of motion, QuickDASH, or any other applicable metric for a particular musculoskeletal anomaly identified within the individual. In some embodiments, the ongoing monitoring includes additional scans, such as acquired at 102, which can allow for some embodiments to analyze and make additional recommendations for treatment or care based on any changes to an individual's condition.

It should be noted that in various embodiments, certain features of method 100 may be omitted, repeated, and/or completed in a different order (included in parallel or at substantially the same time). For example, obtaining a 3D scan 102 can include having obtained a 3D scan, such that the 3D scan is performed by a different entity and stored or transmitted to a system for analysis. Additionally, multiple analyzing 3D representation 104 features can be used, such that different parameters or different areas or regions of the scan can be analyzed as necessary for the determining a deformity, break, and/or other anomaly. Similarly, multiple treatment recommendations 106 can be made, should multiple anomalies be discovered. In many embodiments, the analysis 104 and recommendation 106 features can be accomplished simultaneously and/or with a single machine learning model.

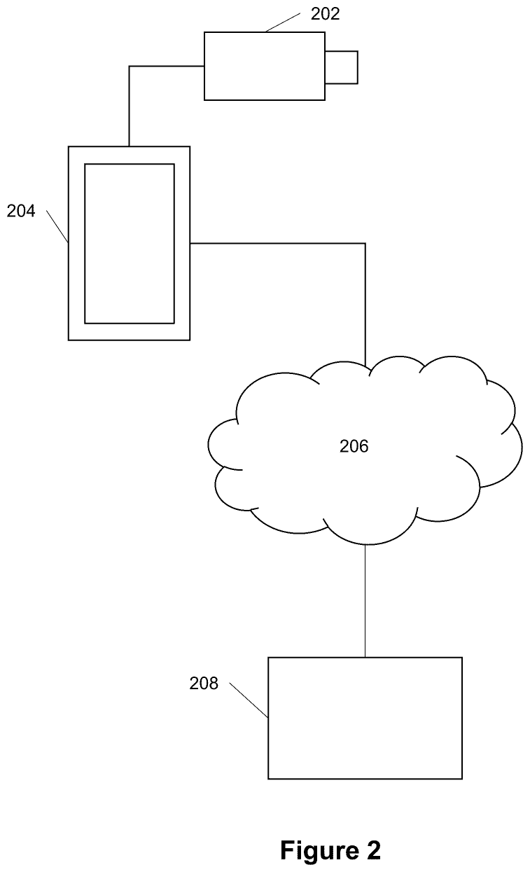

Turning to FIG. 2, an exemplary system for detecting skeletal anomalies is illustrated. In many embodiments, a 3D scan is accomplished using a 3D scanning device 202. In many embodiments, the 3D scanning device 202 is portable, such that it can be moved and/or deployed easily. In many embodiments, the 3D scanning device 202 is capable of obtaining scans of a human body without emitting ionizing or other damaging radiation, such as a white light scanning camera or a LiDAR-enabled camera.

Many embodiments deploy the 3D scanning device 202 is in communication with a computing device 204. In certain embodiments, the computing device 204 is capable of storing and transmitting data, including transmitting data over a network. In certain embodiments, the 3D scanner (e.g., white light scanner or LiDAR system) is innate to the device, while some embodiments utilize deploy the 3D scanner peripheral device attached to the computing device 204. In certain embodiments, the communication between the 3D scanning device 202 and the computing device 204 is a wired communication, such as via USB, serial, audio, RCA, HDMI, coaxial, and/or other form of wired communication, while some embodiments use wireless communication, such as Bluetooth, wi-fi, RF, or other wireless communication systems.

In many embodiments a computing device 204 is a mobile or portable device, such as a mobile phone, tablet, laptop/notebook computer, to allow portability and ease of operation outside of a medical facility. Various embodiments analyze an acquired 3D scan for skeletal anomalies (e.g., broken bone, deformity, etc.) locally, while in some embodiments, computing device 204 is connected to a network 206 (e.g., wired or wireless) to allow communication of a 3D scan to other devices, such as a server 208.

In embodiments connected to a server 208 allow for a higher processing power and/or storage capacity for 3D scans. In such embodiments, a server 208 analyzes such scans to diagnose and/or make recommendations for treatment and/or ongoing care.

### Embodiments are Capable of Identifying Anomalies

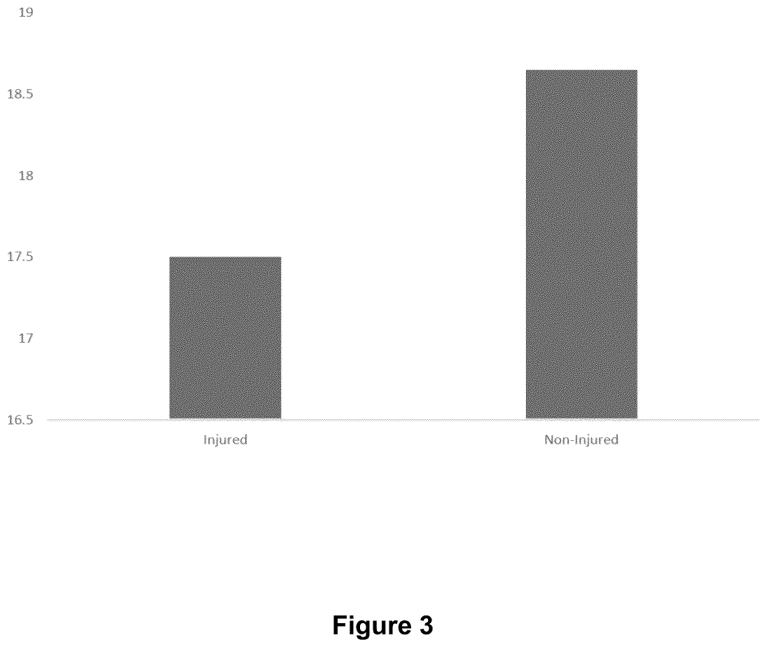

Turning to FIG. 3, exemplary results of scores for injured and non-injured shoulders are illustrated. As seen in FIG. 3, many embodiments are capable of identifying or discerning an injury or anomaly based on the scans obtained from an individual.

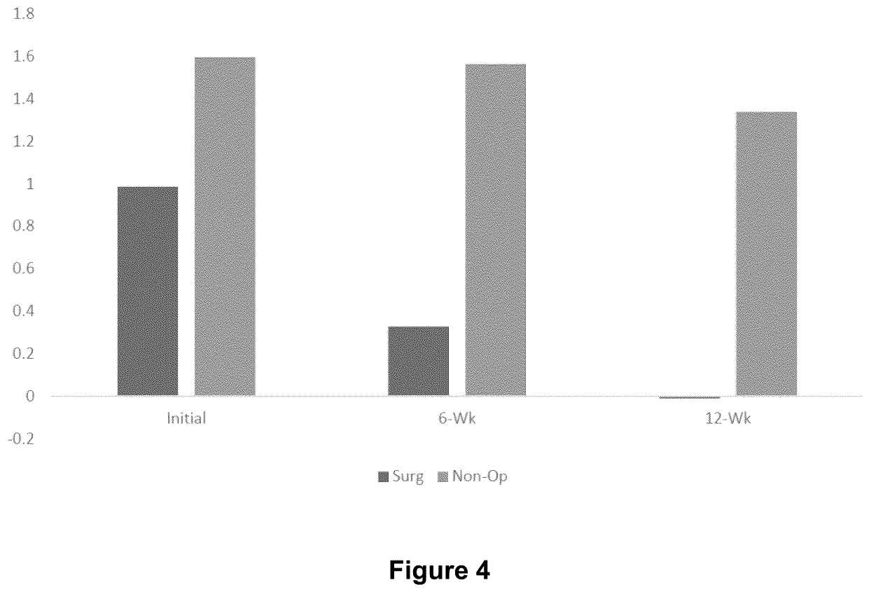

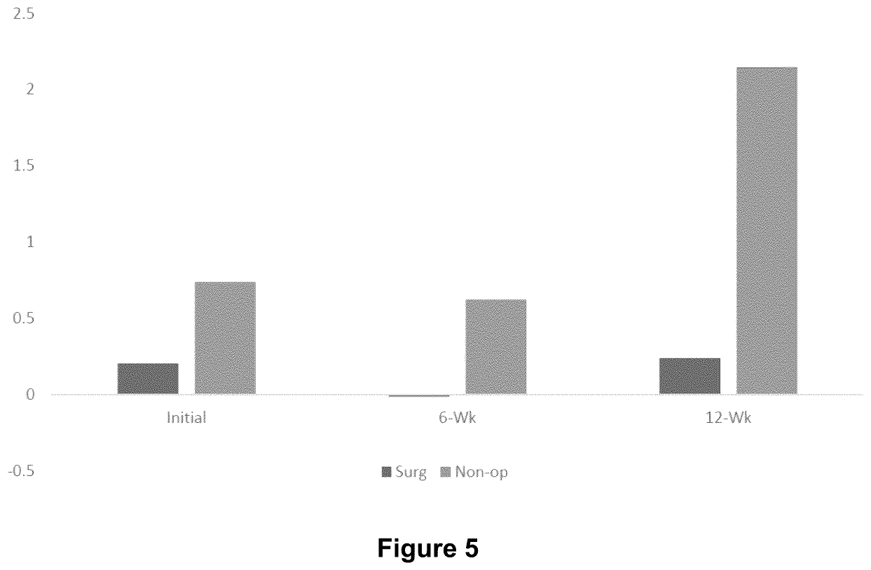

Additionally, many embodiments are capable of identifying persistent deformities over time. Turning to FIG. 4, exemplary results of a shoulder deformity identified in surgical and non-surgical patients over time illustrates that many embodiments are capable of identifying a persistent deformity in non-surgical patients, while normal anatomies are restored in the surgical patients. In contrast, FIG. 5 illustrates hand measurements for shoulder deformities for surgical and non-surgical patients. As seen in in FIG. 5, hand measurements are not sensitive enough to show a difference in improvement of the deformity over time.

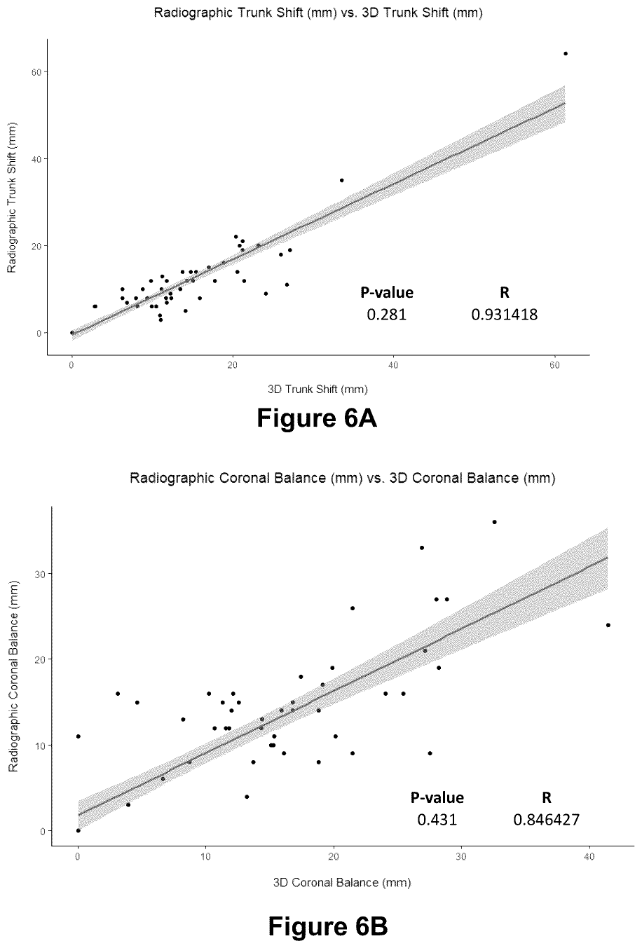

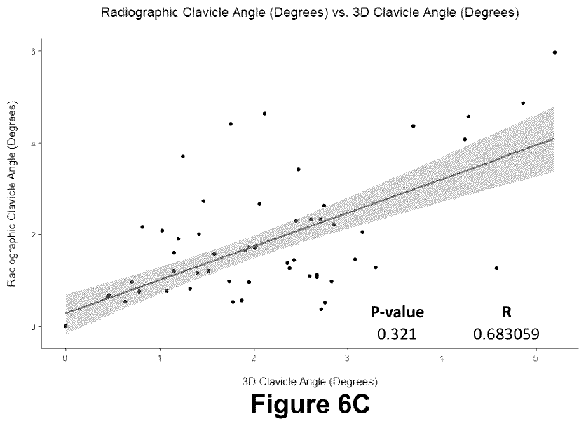

Turning to FIGS. 6A-6C, various embodiments of 3D-scanning based detection of musculoskeletal anomalies correlate to radiographic (e.g., X-ray based) detections. In particular, various embodiments show correlations between trunk shift (FIG. 6A), coronal balance (FIG. 6B), and clavicle angle (FIG. 6C). Additionally, FIGS. 6D-6I illustrate how certain embodiments of 3D-scanning based detection (FIGS. 6D-6F) of angle of trunk rotation correlate to radiographic measurements of cobb angle better than traditional means using a scoliometer (FIG. 6G-6I), including overall (FIGS. 6D and 6G), thorax (FIGS. 6E and 6H) and lumbar (FIGS. 6F and 6I). FIGS. 6A-6I illustrate that various embodiments correlate to radiography and supplant radiography as a means to detect musculoskeletal anomalies.

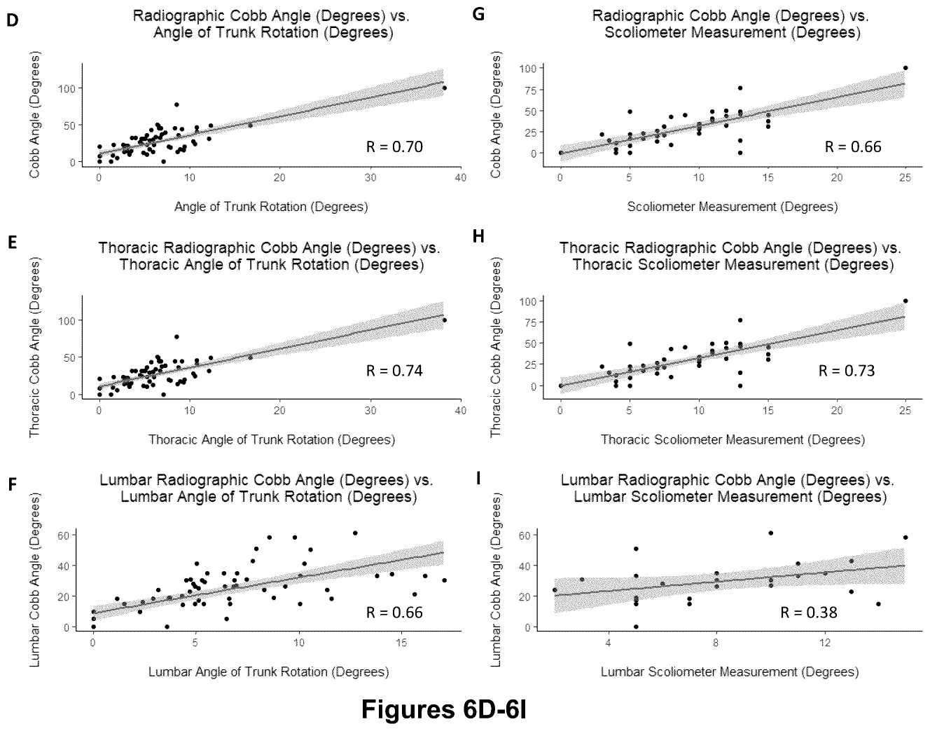

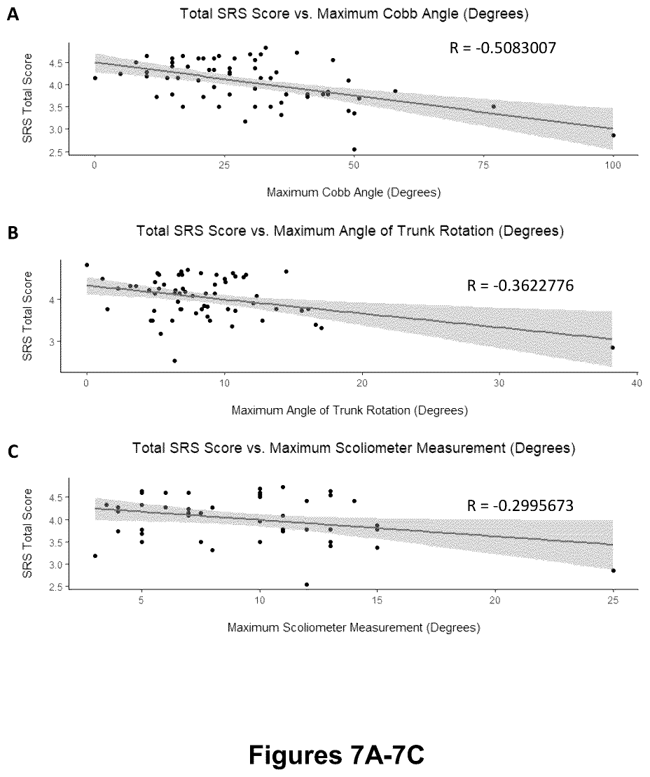

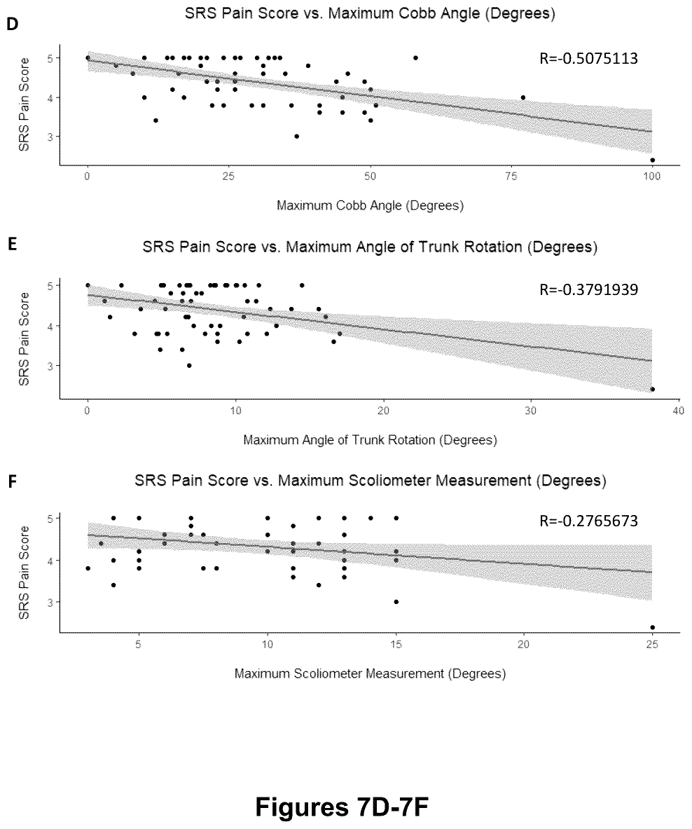

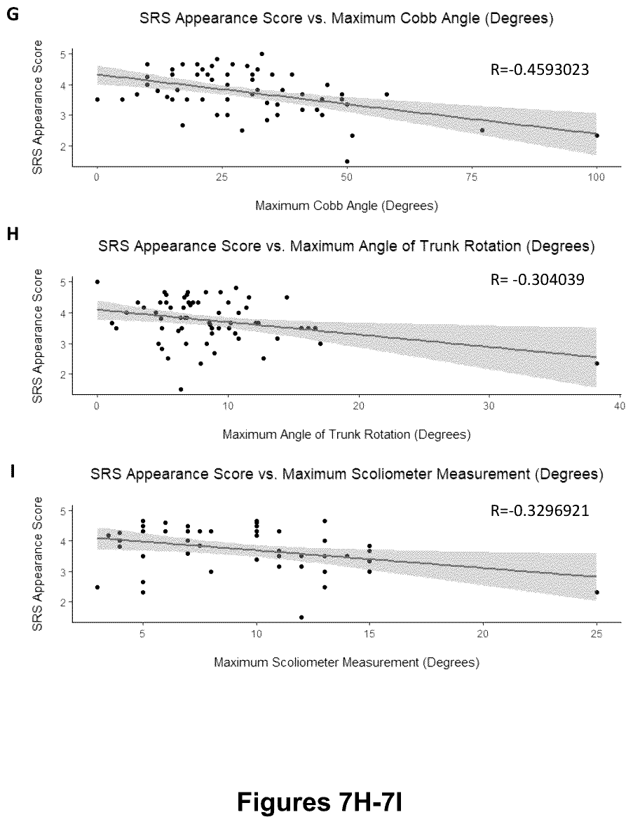

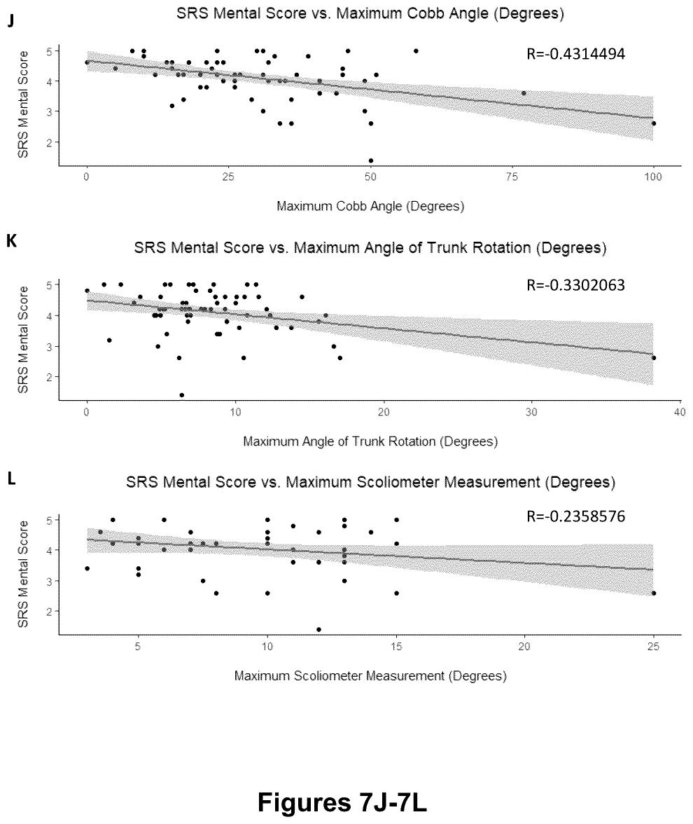

Additionally, FIGS. 7A-7L illustrate correlations patient-reported outcome measures (PROMs) in terms of the SRS scores. Specifically, FIGS. 7A-7C illustrate correlations with total SRS scores versus various methods of measuring various anomalies, FIGS. 7D-7F illustrate correlations with SRS pain scores, FIGS. 7G-7I illustrate correlations with SRS appearance scores, and FIGS. 7J-7L illustrate correlations with SRS mental scores versus maximum cobb angle (FIGS. 7A, 7D, 7G, and 7J), maximum angle of trunk rotation (FIGS. 7B, 7E, 7H, and 7K), and maximum scoliometer measurement (FIGS. 7C, 7F, 7I, and 7L). FIGS. 7A-7L illustrate that various embodiments are comparable to radiographic and scoliometer measurements.

Additionally, various embodiments are capable of making treatment suggestions or recommendations. As seen in Tables 1A-1G illustrate information regarding various subjects, including demographic information, SRS Scores, and various scores obtained from radiography, 3D scans, and output including recommendations for intervention. A full description of the headings are illustrated in Table 2.

### EXEMPLARY EMBODIMENTS

Although the following embodiments provide details on certain embodiments of the inventions, it should be understood that these are only exemplary in nature, and are not intended to limit the scope of the invention.

### Example 1: Mobile Device Based 3D Scanning Accurately Captures Deformity in Adolescent Idiopathic Scoliosis

BACKGROUND: Diagnosis and management of adolescent idiopathic scoliosis (AIS) currently relies on in-person clinical and radiographic examination. Characterization of deformity in adolescent idiopathic scoliosis (AIS) is typically described by metrics such as trunk shift, coronal balance, clavicle angle, and angle of trunk rotation. Structural analysis of 3D scans of patients in forward bend position provides an opportunity to further characterize this deformity. White-light 3D scanning (WL3D) can generate high quality 3D representations of surface anatomy using a mobile device. It was hypothesized that WL3D would provide accurate deformity assessments compared to scoliometer and radiographic measurements. Additionally, this study describes a novel measurement method for AIS deformity characterization.

Methods: Prospective enrollment included patients 10 to 18 years old with AIS, who had a scoliosis radiograph within 30 days of clinic presentation and no history of spinal surgery. 3D scans were taken in the upright and Adams forward bend positions, after which patients completed the SRS-30. Image processing software was used to make 3D measurements of trunk shift, coronal balance, and clavicle angle in upright position and angle of largest trunk rotation (ATR) as detected in the lumbar and thoracic spine in bending position. Modeling software was used to make axial slices of the torso orthogonal to the line of curvature created by the patient's back. The slice passing through the area with the largest angle of trunk rotation was analyzed. A line representing the “horizon” was drawn axially from left to right mid-sagittal line, and a perpendicular line was drawn from the posterior axis of rotation of the trunk to the horizon line. Bilateral circumferential distance along the posterior edge from mid-sagittal line to the axis of rotation and the area created by each posterior quadrant was measured to quantify asymmetry. 3D trunk shift, coronal balance, clavicle angle were compared to their analogous radiographic measurements, and ATR was correlated to cobb angle from radiographs and angle of trunk rotation as measured by a scoliometer (SM).

RESULTS: Sixty-three patients were included in the study. Mean coronal Cobb angle was 33.1°, range: 10 to 100 degrees. Correlations between the clavicle angle, shoulder height, trunk shift, and coronal balance measurements taken from 3D topographical and radiographic measurements were 0.95, 0.85, and 0.71 respectively. Correlations between cobb angle and 3D ATR were 0.7 overall (FIG. 6D), 0.73 in the thoracic spine (FIG. 6E), and 0.66 in the lumbar spine (FIG. 6F). Correlations between cobb angle and SM were 0.64 overall (FIG. 6G), 0.73 in the thoracic spine (FIG. 6H), and 0.38 in the lumbar spine (FIG. 6I). A univariate model more accurately predicts cobb angle as a function of the 3D ATR (p<0.01) compared to a univariate model that predicts cobb angle as a function of scoliometer measurement (p=0.154).

Patients with surgical curves (CM>40°) had significantly larger axial area asymmetry. Patients with at least bracing range curves (CM>20°) had significantly larger axial area asymmetry compared to those with Cobb angle <20°. CM and total SRS score had a correlation of −0.5. Difference in quadrant area had a correlation of −0.53 with total SRS. Difference in circumferential distance had a correlation of −0.5 with total SRS.

CONCLUSION: Obtaining a 3D scan of patients with AIS offers an opportunity to further characterize deformity beyond currently accepted metrics. Portable 3D scanning identifies clinically relevant scoliotic deformity and is more predictive of radiographic cobb angle than scoliometer examination. This new modality can facilitate scoliosis screening and monitoring without in-person clinic visits or radiation exposure.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

TABLE 1A

| subjectid | new | age | gender | srs_available | srs_pain_hm | srs_appearance_hm | srs_activity_hm |
|---|---|---|---|---|---|---|---|
| 1 | N | 16 | M | Y | 4 | 4.25 | 3.5 |
| 2 | Y | 13 | F | Y | 3.6 | 3.5 | 3.8 |
| 3 | N | 14 | F | Y | 5 | 4.5 | 4.2 |
| 4 | N | 12 | M | Y | 4 | 2.666666667 | 3.8 |
| 5 | N | 16 | F | Y | 4.2 | 3 | 4.2 |
| 7 | N | 12 | F | Y | 4.6 | 4.166666667 | 4.6 |
| 8 | N | 11 | M | Y | 5 | 3.6 | 3.8 |
| 9 | N | 14 | M | Y | 4.6 | 4 | 4.6 |
| 10 | N | 11 | M | Y | 3.4 | 3.8 | 3.2 |
| 11 | N | 13 | F | Y | 5 | 3.67 | 3.2 |
| 12 | N | 15 | F | Y | 4.6 | 4.33 | 4.6 |
| 13 | N | 13 | F | Y | 5 | 4.6 | 4.4 |
| 14 | N | 12 | F | Y | 4.2 | 4.17 | 3.4 |
| 15 | N | 16 | M | N | | | |
| 16 | N | 12 | F | Y | 4.4 | 4.6 | 4.6 |
| 17 | N | 14 | F | N | | | |
| 18 | N | 12 | M | Y | 5 | 3.83 | 4 |
| 19 | N | 16 | F | Y | 3.4 | 1.5 | 4 |
| 21 | N | 18 | F | Y | 3.8 | 2.33 | 4.6 |
| 22 | N | 13 | M | Y | 5 | 3.4 | 4.2 |
| 23 | N | 11 | M | N | | | |
| 24 | N | 13 | F | Y | 4 | 3 | 4.2 |
| 25 | N | 17 | M | Y | 3.8 | 2.5 | 3.2 |
| 26 | N | 16 | F | Y | 4.4 | 3.17 | 4 |
| 27 | N | 13 | F | Y | 3.8 | 3 | 4 |
| 29 | N | 13 | F | Y | 4.8 | 4.33 | 4.6 |
| 30 | N | 15 | F | Y | 4.4 | 3.5 | 3.4 |
| 31 | N | 16 | F | Y | 4.2 | 3.33 | 3.6 |
| 32 | N | 11 | F | Y | 5 | 4.5 | 4.6 |
| 33 | N | 12 | M | Y | 4.8 | 3.5 | 4.4 |
| 34 | N | 11 | F | Y | 4.2 | 3.5 | 4 |
| 35 | N | 17 | F | Y | 3.6 | 3.67 | 4.6 |
| 36 | N | 13 | F | Y | 3.8 | 3.67 | 4.4 |
| 37 | N | 16 | F | Y | 4 | 2.5 | 4.4 |
| 38 | N | 14 | M | Y | 5 | 4 | 4.4 |
| 39 | N | 13 | F | Y | 2.4 | 2.33 | 4.2 |
| 40 | N | 17 | M | Y | 3.6 | 3.5 | 4.2 |
| 41 | N | 15 | F | Y | 4.6 | 4 | 4.6 |
| 42 | N | 13 | F | Y | 5 | 4.67 | 4.6 |
| 43 | N | 16 | F | Y | 3.8 | 4.33 | 2.8 |
| 44 | N | 13 | F | Y | 3.8 | 3.17 | 4.2 |
| 45 | N | 13 | M | Y | 5 | 4.83 | 4 |
| 46 | N | 12 | M | Y | 5 | 4 | 3.8 |
| 47 | N | 14 | M | Y | 5 | 4.67 | 4.6 |
| 48 | N | 16 | F | Y | 4.4 | 3.67 | 4.4 |
| 50 | N | 16 | F | Y | 5 | 4.67 | 4.6 |
| 51 | N | 15 | M | Y | 4.8 | 4.17 | 4.2 |
| 52 | N | 15 | F | Y | 3.8 | 3.33 | 3.8 |
| 53 | N | 16 | F | Y | 4.8 | 4.33 | 3.2 |
| 55 | N | 17 | M | Y | 3 | 3.83 | 4.4 |
| 56 | N | 14 | F | Y | 3.8 | 4.33 | 4.4 |
| 57 | N | 18 | F | Y | 4.2 | 3.5 | 3.8 |
| 59 | Y | 12 | M | Y | 5 | 2.83 | 4.8 |
| 61 | Y | 16 | F | Y | 5 | 4.5 | 4.6 |
| 62 | Y | 13 | F | Y | 5 | 3.5 | 4.6 |
| 63 | Y | 13 | M | Y | 5 | 3.5 | 4.6 |
| 65 | Y | 12 | M | Y | 4.6 | 3.67 | 4.6 |
| 66 | Y | 13 | F | Y | 5 | 5 | 4.4 |
| 67 | Y | 15 | F | Y | 5 | 4.33 | 4.2 |
| 68 | Y | 16 | M | Y | 4.4 | 4.17 | 4.4 |
| 69 | Y | 14 | F | Y | 3.8 | 3 | 3.8 |
| 71 | Y | 11 | F | Y | 5 | 4.67 | 4.4 |
| 72 | Y | 14 | M | Y | 4.4 | 4.33 | 4.6 |
| 73 | Y | 15 | M | Y | 4.6 | 3.83 | 4 |
| 74 | Y | 15 | M | Y | 5 | 3.5 | 4.4 |

TABLE 1B

| subjectid | srs_mental_hm | srs_satisfaction_hm | srs_tscore_hm | srs_hmscore | maxATR | maxcobb |
|---|---|---|---|---|---|---|
| 1 | 5 | | 4.18 | | 7.125 | 10 |
| 2 | 3 | 3 | 3.41 | 5 | 16.641 | 49 |
| 3 | 4.6 | 4.5 | 4.59 | | 11.57 | 21 |
| 4 | 3.4 | 4 | 3.5 | 4.666666667 | 8.947 | 17 |
| 5 | 3.8 | 3 | 3.772727273 | 4 | 6.812 | 26 |
| 7 | 5 | 4 | 4.545454545 | 3.666666667 | 11.383 | 31 |
| 8 | 4.6 | 3 | 4.14 | 4.33 | 8.619 | 14 |
| 9 | 4 | 3.5 | 4.23 | 4.67 | 4.51 | 26 |
| 10 | 4.2 | 4.5 | 3.73 | 1 | 4.89 | 12 |
| 11 | 5 | 1 | 3.86 | 1 | 8.55 | 58 |
| 12 | 4 | 3 | 4.27 | 4.33 | 6.993 | 35 |
| 13 | 5 | 4 | 4.68 | 6 | 6.837 | 30 |
| 14 | 4.2 | 3.5 | 3.95 | 4 | 6.59 | 23 |
| 15 | | | | | 4.842 | 28 |
| 16 | 5 | 4 | 4.59 | 6 | 5.269 | 23 |
| 17 | | | | | 5.97 | 13 |
| 18 | 4 | 3.5 | 4.14 | 3 | 6.712 | 32 |
| 19 | 1.4 | 2 | 2.54 | 5 | 6.386 | 50 |
| 21 | 4.2 | 3 | 3.68 | 5 | 7.913 | 51 |
| 22 | 2.6 | 3 | 3.5 | 3 | 6.212 | 34 |
| 23 | | | | | 7.459 | 45 |
| 24 | 4.2 | 3 | 3.77 | 4.67 | 8.366 | 45 |
| 25 | 3.4 | 2.5 | 3.18 | 5 | 5.392 | 29 |
| 26 | 3.6 | 3 | 3.77 | 4.33 | 13.785 | 44 |
| 27 | 2.6 | 3 | 3.32 | 3.67 | 17.075 | 36 |
| 29 | 4.8 | 5 | 4.72 | 5 | 7.308 | 39 |
| 30 | 3.8 | 3 | 3.73 | 3.33 | 15.604 | 21 |
| 31 | 2.6 | 3 | 3.36 | 4.3 | 10.552 | 50 |
| 32 | 4.4 | 4.5 | 4.59 | 6 | 6.658 | 15 |
| 33 | 4.4 | 4 | 4.23 | 4 | 6.466 | 5 |
| 34 | 3.2 | 4 | 3.77 | 3.33 | 1.479 | 15 |
| 35 | 3.6 | 3 | 3.77 | 2.67 | 10.243 | 41 |
| 36 | 4.2 | 3 | 3.91 | 4 | 12.106 | 31 |
| 37 | 3.6 | 3 | 3.5 | 6 | 12.737 | 77 |
| 38 | 4.2 | 4 | 4.36 | 5.33 | 9.309 | 26 |
| 39 | 2.6 | 4 | 2.86 | 4.33 | 38.213 | 100 |
| 40 | 4.4 | 3 | 3.83 | 3 | 8.773 | 45 |
| 41 | 5 | 4 | 4.55 | 1 | 10.807 | 46 |
| 42 | 3.8 | 5 | 4.59 | 6 | 9.441 | 20 |
| 43 | 3 | 4 | 3.5 | 4 | 4.795 | 31 |
| 44 | 4 | 3 | 3.73 | 4 | 10.777 | 41 |
| 45 | 4.6 | 5 | 4.64 | 6 | 10.59 | 24 |
| 46 | 5 | 3 | 4.27 | 3 | 2.259 | 10 |
| 47 | 4.2 | 4.5 | 4.59 | 5.33 | 6.94 | 27 |
| 48 | 4 | 4 | 4.09 | 4.67 | 12.332 | 49 |
| 50 | 4.2 | 4.5 | 4.64 | 5 | 5.127 | 17 |
| 51 | 5 | 3 | 4.36 | 3 | 5.594 | 31 |
| 52 | 3.4 | 4 | 3.59 | 3 | 8.789 | 36 |
| 53 | 4.2 | 4 | 4.09 | 5 | 7.717 | 20 |
| 55 | 4.2 | 3 | 3.77 | 3.33 | 6.862 | 37 |
| 56 | 4.4 | 5 | 4.32 | 5 | 3.13 | 22 |
| 57 | 4 | 3 | 3.77 | 4.67 | 16.121 | 26 |
| 59 | 4 | 3.5 | 4.14 | 6 | 4.958 | 34 |
| 61 | 4.6 | 4.5 | 4.68 | 6 | 14.51 | 32 |
| 62 | 4.6 | 3.5 | 4.41 | 5 | 4.953 | 15 |
| 63 | 4.6 | 3.5 | 4.41 | 5 | 10.058 | 18 |
| 65 | 5 | 4.5 | 4.5 | 5.33 | 1.151 | 8 |
| 66 | 4.8 | 5 | 4.82 | 3.33 | 0 | 33 |
| 67 | 4.4 | 4.5 | 4.5 | 5 | 10.027 | 15 |
| 68 | 4.6 | 3.5 | 4.32 | 6 | 3.56 | 23 |
| 69 | 4 | 2.5 | 3.5 | 5 | 4.678 | 24 |
| 71 | 4.8 | 4 | 4.64 | 1.33 | 8.311 | 10 |
| 72 | 4.2 | 3 | 4.27 | 6 | 5.272 | 26 |
| 73 | 4.2 | 4 | 4.14 | 5 | 6.378 | 16 |
| 74 | 4.6 | 5 | 4.14 | 3 | 9.313 | 0 |

TABLE 1C

| subjectid | maxsm | TS_XR | CB_XR | CLAV_ANG_XR | TS_3D | CB_3D | CLAV_ANG_3D | LATR |
|---|---|---|---|---|---|---|---|---|
| 1 | 4 | 0 | 0 | 2.17 | 0 | 0 | 0.815 | 0 |
| 2 | 13 | 21 | 19 | 4.58 | 21.24497284 | 19.87635516 | 4.279 | 5.01 |
| 3 | 7 | 8 | 12 | 0.964 | 7.924862669 | 14.33294746 | 0.702 | 11.57 |
| 4 | 5 | 6 | 15 | 4.855 | 2.902637055 | 4.658700394 | 4.858 | 0 |
| 5 | 13 | 0 | 0 | 0 | 0 | 0 | 0 | 6.812 |
| 7 | 10 | 6 | 6 | 0.975 | 9.934577955 | 6.679008203 | 2.824 | 11.383 |
| 8 | 7 | 7 | 12 | 1.291 | 6.815532032 | 10.71012176 | 3.291 | 0 |
| 9 | 7 | 13 | 13 | 1.567 | 11.22517782 | 14.44199306 | 1.58 | 0 |
| 10 | 4 | 12 | 12 | 1.163 | 11.80346016 | 11.80346016 | 1.397 | 0 |
| 11 | 15 | 10 | 14 | 2.323 | 11.09939383 | 11.98393013 | 2.603 | 8.55 |
| 12 | 6 | 16 | 14 | 0.523 | 18.92617599 | 16.82326754 | 1.779 | 6.993 |
| 13 | 10 | 10 | 10 | 1.437 | 13.4922324 | 15.2979696 | 2.422 | 6.837 |
| 14 | 10 | 0 | 0 | 4.413 | 0 | 0 | 1.75 | 6.59 |
| 15 | 6 | 5 | 9 | 3.422 | 14.15531665 | 16.08457166 | 2.473 | 4.842 |
| 16 | 6 | 8 | 8 | 1.198 | 9.329931401 | 8.728582876 | 1.148 | 4.34 |
| 17 | | 12 | 16 | 1.909 | 9.862989289 | 10.22791989 | 1.196 | 0 |
| 18 | | 8 | 0 | 2.67 | 12.35095681 | 0 | 2.059 | 5.348 |
| 19 | 12 | 14 | 11 | 0 | 14.74143826 | 20.18208194 | 0 | 5.105 |
| 21 | 5 | 4 | 10 | 0 | 10.9647922 | 15.08389914 | 0 | 7.913 |
| 22 | 10 | 14 | 0 | 1.383 | 13.73430457 | 0 | 2.357 | 3.141 |
| 23 | 9 | 20 | 0 | 1.267 | 20.87553957 | 0 | 4.579 | 7.459 |
| 24 | 15 | 8 | 0 | 5.964 | 11.70546492 | 0 | 5.194 | 7.763 |
| 25 | 3 | 0 | 14 | 0 | 0 | 18.83037194 | | 5.392 |
| 26 | 12 | 12 | 0 | 0 | 14.29768085 | 0 | 0 | 13.785 |
| 27 | 8 | 14 | 14 | 1.599 | 15.41829779 | 15.90204688 | 1.147 | 17.075 |
| 29 | 11 | 10 | 0 | 1.269 | 8.819908219 | 0 | 2.378 | 0 |
| 30 | | 0 | 16 | 4.364 | 0 | 12.15131685 | 3.694 | 15.604 |
| 31 | 15 | 22 | 33 | 1.741 | 20.38831024 | 26.90327988 | 2.028 | 10.552 |
| 32 | 5 | 8 | 15 | 0.982 | 6.300114966 | 12.60022993 | 1.735 | 6.658 |
| 33 | | 0 | 0 | 0 | 0 | 0 | 0 | 6.466 |
| 34 | 5 | 10 | 13 | 1.66 | 6.29109396 | 8.241333088 | 1.909 | 1.479 |
| 35 | 11 | 6 | 16 | 2.631 | 2.825482302 | 3.142878148 | 2.748 | 10.243 |
| 36 | | 0 | 0 | 2.319 | 0 | 0 | 2.703 | 0 |
| 37 | 13 | 14 | 0 | 2.214 | 20.53690846 | 0 | 2.851 | 12.737 |
| 38 | | 9 | 9 | 2.093 | 24.09674364 | 21.4791491 | 1.025 | 9.309 |
| 39 | 25 | 64 | 27 | 2.296 | 61.36533181 | 28.8805635 | 2.447 | 9.791 |
| 40 | | 15 | 17 | 1.698 | 17.05100692 | 19.14528879 | 2.014 | 8.773 |
| 41 | 13 | 3 | 3 | 1.72 | 11.02200543 | 3.91483271 | 1.945 | 6.336 |
| 42 | | 0 | 0 | 1.456 | 0 | 0 | 3.077 | 0 |
| 43 | 7.5 | 0 | 0 | 0.768 | 0 | 0 | 1.071 | 3.667 |
| 44 | 11 | 9 | 16 | 0.67 | 12.2599804 | 25.46303622 | 0.462 | 4.52 |
| 45 | | 0 | 0 | 0 | 0 | 0 | 0 | 4.295 |
| 46 | 4 | 0 | 0 | 0 | 0 | 0 | 0 | 2.259 |
| 47 | 10 | 20 | 26 | 1.999 | 23.19755968 | 21.45130731 | 1.419 | 6.94 |
| 48 | 11 | 0 | 8 | 0.509 | 0 | 18.84031332 | 2.751 | 5.543 |
| 50 | 5 | 7 | 19 | 0 | 11.7715736 | 28.25177665 | 0 | 5.127 |
| 51 | | 0 | 15 | 0.82 | 0 | 11.31510133 | 1.322 | 4.715 |
| 52 | | 0 | 0 | 2.056 | 0 | 0 | 3.156 | 5.285 |
| 53 | 7 | 0 | 12 | 1.116 | 0 | 11.58589218 | 2.668 | 3.687 |
| 55 | 15 | 6 | 11 | 4.068 | 8.164482176 | 0 | 4.236 | 0 |
| 56 | 5 | 12 | 8 | 2.72 | 17.74399336 | 13.73442682 | 1.463 | 2.915 |
| 57 | 11 | 35 | 36 | 0.762 | 33.56457215 | 32.60558437 | 0.785 | 16.121 |
| 59 | | 12 | 15 | 0.373 | 15.07928639 | 16.78037322 | 2.715 | 4.958 |
| 61 | 10 | 19 | 24 | 1.095 | 27.0488814 | 41.50031797 | 2.588 | 14.51 |
| 62 | 12 | 6 | 18 | 4.641 | 10.44332837 | 17.42730421 | 2.11 | 4.953 |
| 63 | 14 | 0 | 0 | 0 | 0 | 0 | 0 | 10.058 |
| 65 | | 8 | 4 | 0 | 15.86992094 | 13.20190717 | 0 | 1.151 |
| 66 | | 0 | 0 | 0.958 | 0 | 0 | 1.946 | 0 |
| 67 | 10 | 0 | 0 | 0.642 | 0 | 0 | 0.444 | 10.027 |
| 68 | 3.5 | 0 | 0 | 0 | 0 | 0 | 0 | 3.56 |
| 69 | 13 | | 21 | 1.076 | | 27.13072059 | 2.671 | 4.678 |
| 71 | 13 | 18 | 9 | 3.703 | 25.95944214 | 27.55595552 | 1.241 | 8.311 |
| 72 | 8 | 19 | 27 | 0.564 | 21.17693288 | 28.02969318 | 1.868 | 0 |
| 73 | 7.5 | 12 | 11 | 1.204 | 21.36180203 | 15.38018986 | 1.517 | 6.378 |
| 74 | | 11 | 16 | 0.527 | 26.67779104 | 24.06803542 | 0.638 | 2.403 |

TABLE 1D

| subjectid | LACA | LSM_YN | LSM | L_DISP_DIFF | L_DISP_DIFF_PER | L_DIS_DIF | L_DIS_DIF_PER |
|---|---|---|---|---|---|---|---|
| 1 | 10 | N | | 1.504230882 | 0.006302697 | 3.863254412 | 0.011598614 |
| 2 | 41 | N | | 56.27847919 | 0.206004536 | 56.73723666 | 0.152441722 |
| 3 | 18 | Y | 7 | 16.29488182 | 0.070204833 | 35.18204112 | 0.106135573 |
| 4 | 5 | N | | 1.743584914 | 0.00591863 | 5.86569681 | 0.011710865 |
| 5 | 26 | Y | 8 | 0.726511922 | 0.003043552 | 7.769991654 | 0.023500816 |
| 7 | 24 | N | | 21.2614357 | 0.092253874 | 26.22846445 | 0.080734225 |
| 8 | 0 | N | | 15.68015641 | 0.068498678 | 14.22125878 | 0.046581589 |
| 9 | 0 | N | | 0.564648937 | 0.001818036 | 7.394077828 | 0.016670422 |
| 10 | 0 | N | | 6.146666667 | 0.019169207 | 6.031416667 | 0.010486885 |
| 11 | 58 | Y | 15 | 62.15997415 | 0.189811689 | 73.58046034 | 0.147533948 |
| 12 | 35 | N | | 35.76187363 | 0.137430509 | 41.9846312 | 0.123269707 |
| 13 | 30 | Y | 10 | 11.86420859 | 0.053431677 | 8.506941376 | 0.026916507 |
| 14 | 18 | N | | 54.26496529 | 0.233500664 | 34.68271117 | 0.094970832 |
| 15 | 28 | Y | 6 | 25.96717433 | 0.090417254 | 43.74888307 | 0.112298391 |
| 16 | 14 | N | | 3.743604057 | 0.015511989 | 10.81878028 | 0.033030119 |
| 17 | 0 | N | | 18.89019727 | 0.078652435 | 6.931652149 | 0.02204762 |
| 18 | 15 | N | | 24.38236585 | 0.07575611 | 9.914606471 | 0.017623399 |
| 19 | 25 | N | | 32.59560931 | 0.095078791 | 36.16288884 | 0.065616535 |
| 21 | 51 | Y | 5 | 10.89860672 | 0.040388093 | 10.86383775 | 0.023675067 |
| 22 | 24 | N | | 32.50043233 | 0.1249289 | 28.98625947 | 0.053150719 |
| 23 | 31 | Y | 3 | 30.2593242 | 0.133117363 | 33.50617423 | 0.110344812 |
| 24 | 43 | Y | 13 | 9.430406326 | 0.039004981 | 5.051258805 | 0.01326942 |
| 25 | 29 | N | | 16.3154374 | 0.049374467 | 21.636631 | 0.04590789 |
| 26 | 33 | N | | 11.38151152 | 0.045936288 | 9.772924561 | 0.031447498 |
| 27 | 30 | Y | 8 | 4.540659161 | 0.017258835 | 25.43437874 | 0.073557058 |
| 29 | 0 | N | | 0 | 0 | 8.060943316 | 0.021434501 |
| 30 | 21 | N | | 29.32223932 | 0.135671493 | 52.01316773 | 0.173559374 |
| 31 | 50 | N | | 21.30539178 | 0.067647266 | 35.48913909 | 0.082516708 |
| 32 | 15 | Y | 5 | 9.199063903 | 0.041117604 | 4.049216735 | 0.014176342 |
| 33 | 5 | N | | 19.21093829 | 0.077033517 | 25.97050038 | 0.07021429 |
| 34 | 15 | N | | 0 | 0 | 1.54238175 | 0.003765567 |
| 35 | 41 | Y | 11 | 17.7066485 | 0.069627543 | 35.64908522 | 0.098837573 |
| 36 | 0 | N | | 4.559925735 | 0.020154664 | 8.934648333 | 0.028161664 |
| 37 | 61 | Y | 10 | 1.011694805 | 0.003840951 | 15.8752372 | 0.045281618 |
| 38 | 26 | N | | 0.488113036 | 0.001981692 | 6.10243411 | 0.017728203 |
| 39 | 58 | N | | 4.13593427 | 0.020393558 | 28.896304 | 0.095462218 |
| 40 | 19 | N | | 10.09739053 | 0.040991644 | 20.41951142 | 0.061206709 |
| 41 | 35 | Y | 12 | 2.807735553 | 0.011283398 | 7.579493728 | 0.022839147 |
| 42 | 0 | N | | 3.2150265 | 0.012132257 | 1.559379347 | 0.004450459 |
| 43 | 19 | N | | 3.696018735 | 0.014372193 | 7.770908145 | 0.024053947 |
| 44 | 30 | N | | 13.99465479 | 0.051224488 | 15.23409443 | 0.038140751 |
| 45 | 20 | N | | 31.50821789 | 0.125896971 | 39.8777763 | 0.090948718 |
| 46 | 10 | N | | 9.426721978 | 0.03684413 | 5.745804843 | 0.016178329 |
| 47 | 27 | Y | 10 | 5.533463921 | 0.021093133 | 5.245947421 | 0.01506116 |
| 48 | 35 | Y | 8 | 19.08890691 | 0.078198836 | 2.037590662 | 0.005344866 |
| 50 | 17 | Y | 5 | 18.09250921 | 0.046871786 | 25.39826825 | 0.039271121 |
| 51 | 31 | N | | 48.74906218 | 0.174565107 | 42.64453461 | 0.09919571 |
| 52 | 30 | N | | 21.2554085 | 0.075356932 | 24.59286158 | 0.063634854 |
| 53 | 19 | Y | 5 | 2.807778831 | 0.009799021 | 6.680121652 | 0.014100921 |
| 55 | 0 | N | | 10.66718543 | 0.03986552 | 12.16083037 | 0.032911696 |
| 56 | 18 | N | | 5.973138034 | 0.025856069 | 4.752525111 | 0.013951422 |
| 57 | 33 | Y | 11 | 63.53204553 | 0.229201604 | 41.34224181 | 0.101495965 |
| 59 | 26 | N | | 21.65814906 | 0.083214034 | 21.3853986 | 0.058285609 |
| 61 | 34 | N | | 35.09703407 | 0.159304324 | 29.7015746 | 0.090602519 |
| 62 | 15 | Y | 7 | 22.35447652 | 0.095443552 | 25.31237264 | 0.070676069 |
| 63 | 15 | Y | 14 | 10.20969644 | 0.043613621 | 0.639318146 | 0.001950698 |
| 65 | 18 | N | | 3.930267734 | 0.012485051 | 4.288307051 | 0.010917943 |
| 66 | 0 | N | | 1.670471647 | 0.006730617 | 1.996794397 | 0.005914377 |
| 67 | 33 | Y | 5 | 9.9510188 | 0.037428124 | 7.077862158 | 0.020182677 |
| 68 | 0 | N | | 114.3307529 | 0.193825422 | 64096.63734 | 0.989426105 |
| 69 | 23 | Y | 13 | 5.1325827 | 0.019655971 | 9.03527027 | 0.024631179 |
| 71 | 24 | Y | 2 | 17.58394183 | 0.070105517 | 23.37141668 | 0.069146708 |
| 72 | 0 | Y | 5 | 1.672681322 | 0.007005541 | 1.229981681 | 0.003640501 |
| 73 | 26 | N | | 61.29931177 | 0.210081412 | 33.81047529 | 0.091768477 |
| 74 | 16 | N | | 3.046184317 | 0.01075023 | 3.158420089 | 0.008242644 |

TABLE 1E

| subjectid | L_AREA_DIF | L_AREA_DIF_PER | TATR | TACA | TSM_YN | TSM | T_DISP_DIFF |
|---|---|---|---|---|---|---|---|
| 1 | 299.4014663 | 0.016962476 | 7.125 | 0 | Y | 4 | 39.70475183 |
| 2 | 5490.122215 | 0.260824295 | 16.641 | 49 | Y | 13 | 115.6213261 |
| 3 | 3215.54501 | 0.194714588 | 0 | 21 | N | | 10.12752149 |
| 4 | 124.2015028 | 0.00357299 | 8.947 | 17 | Y | 5 | 17.27294815 |
| 5 | 997.7747927 | 0.058985483 | 0 | 0 | Y | 13 | 1.675475764 |
| 7 | 2438.980584 | 0.148634985 | 5.081 | 31 | Y | 10 | 12.61497775 |
| 8 | 1321.446949 | 0.091805111 | 8.619 | 14 | Y | 7 | 10.2023953 |
| 9 | 451.4610454 | 0.015577215 | 4.51 | 26 | Y | 7 | 1.871239441 |
| 10 | 178.4126914 | 0.003712155 | 4.89 | 12 | Y | 4 | 5.368104527 |
| 11 | 10024.32257 | 0.258671301 | 5.787 | 43 | Y | 8 | 10.34858905 |
| 12 | 3630.750167 | 0.224588665 | 5.51 | 17 | Y | 6 | 14.05261653 |
| 13 | 1206.775269 | 0.07922998 | 5.547 | 29 | Y | 10 | 12.95074168 |
| 14 | 3727.299165 | 0.185001706 | 5.996 | 23 | Y | 10 | 0.003816407 |
| 15 | 3176.16698 | 0.140823949 | 0 | 0 | N | | 0.007502345 |
| 16 | 901.680915 | 0.057176577 | 5.269 | 23 | Y | 6 | 11.39837334 |
| 17 | 967.990283 | 0.065380446 | 5.97 | 13 | N | | 9.796839664 |
| 18 | 2901.360538 | 0.059309002 | 6.712 | 32 | N | | 9.550213681 |
| 19 | 7679.155741 | 0.16069271 | 6.386 | 50 | Y | 12 | 40.73533279 |
| 21 | 2025.740666 | 0.062878557 | 6.498 | 49 | Y | 5 | 8.206607678 |
| 22 | 5881.230233 | 0.145226342 | 6.212 | 34 | Y | 10 | 4.160756813 |
| 23 | 2742.176546 | 0.200108912 | 6.764 | 45 | Y | 9 | 14.00268005 |
| 24 | 670.4611013 | 0.03049252 | 8.366 | 45 | Y | 15 | 34.52040666 |
| 25 | 2975.590143 | 0.0890111 | 2.791 | 22 | Y | 3 | 33.25627312 |
| 26 | 1253.230446 | 0.091374246 | 9.092 | 44 | Y | 12 | 18.24344124 |
| 27 | 2400.861583 | 0.134039844 | 5.287 | 36 | N | | 23.70096975 |
| 29 | 201.7236807 | 0.009306246 | 7.308 | 39 | Y | 11 | 18.60652308 |
| 30 | 3971.711508 | 0.292163628 | 0 | 0 | N | | 0.00089634 |
| 31 | 4623.349774 | 0.16634382 | 4.665 | 31 | Y | 15 | 5.893665 |
| 32 | 141.64359 | 0.012108204 | 3.28 | 10 | N | | 0.8388729 |
| 33 | 2527.457475 | 0.114799193 | 0 | 0 | N | | 0.623628627 |
| 34 | 280.5209453 | 0.010538707 | 0 | 0 | Y | 5 | 6.703656076 |
| 35 | 3744.60316 | 0.187323339 | 3.59 | 32 | N | | 2.002549616 |
| 36 | 338.7154806 | 0.021898739 | 12.106 | 31 | N | | 11.80116798 |
| 37 | 1288.682264 | 0.072242346 | 8.567 | 77 | Y | 13 | 5.384301267 |
| 38 | 753.9166201 | 0.040643094 | 7.889 | 19 | N | | 7.551367346 |
| 39 | 1889.785336 | 0.135465267 | 38.213 | 100 | Y | 25 | 99.9953253 |
| 40 | 2025.473297 | 0.123810051 | 6.579 | 45 | N | | 5.136515814 |
| 41 | 491.9237072 | 0.031378799 | 10.807 | 46 | Y | 13 | 12.33287956 |
| 42 | 277.2461497 | 0.015030885 | 9.441 | 20 | N | | 16.96653705 |
| 43 | 421.1082544 | 0.028172464 | 4.795 | 31 | Y | 7.5 | 12.62455213 |
| 44 | 2123.995332 | 0.084667719 | 10.777 | 41 | Y | 11 | 60.91677311 |
| 45 | 4987.943452 | 0.167497487 | 10.59 | 24 | N | | 1.117083433 |
| 46 | 651.0329909 | 0.033460647 | 1.971 | 5 | Y | 4 | 14.22503692 |
| 47 | 1036.039202 | 0.056915963 | 4.645 | 24 | N | | 30.12888513 |
| 48 | 126.4303048 | 0.006015182 | 12.332 | 49 | Y | 11 | 22.11999262 |
| 50 | 6587.921971 | 0.100184388 | 1.248 | 0 | Y | 0 | 0.37746114 |
| 51 | 4641.991842 | 0.164841324 | 5.594 | 29 | N | | 17.7909287 |
| 52 | 2714.217197 | 0.118291543 | 8.789 | 36 | N | | 7.947788524 |
| 53 | 724.7609648 | 0.020519911 | 7.717 | 20 | Y | 7 | 13.62079515 |
| 55 | 366.4262968 | 0.018468882 | 6.862 | 37 | Y | 15 | 4.29954801 |
| 56 | 623.4844582 | 0.034498834 | 3.13 | 22 | Y | 5 | 3.116937082 |
| 57 | 4255.967372 | 0.168637328 | 2.647 | 23 | N | | 22.74191104 |
| 59 | 1652.128586 | 0.079964518 | 1.625 | 23 | N | | 49.27727735 |
| 61 | 2521.82559 | 0.15141411 | 10.383 | 28 | Y | 10 | 15.35283574 |
| 62 | 2067.726725 | 0.103330001 | 4.025 | 32 | Y | 12 | 47.49235832 |
| 63 | 20.60632309 | 0.001252146 | 1.441 | 9 | Y | 5 | 3.475770795 |
| 65 | 690.5895708 | 0.032114851 | 0 | 0 | N | | 2.629267788 |
| 66 | 200.9407234 | 0.011864145 | 0 | 8 | N | | 4.448479346 |
| 67 | 25.30082261 | 0.001414232 | 5.903 | 30 | Y | 10 | 24.93183522 |
| 68 | 35758.351 | 1 | 3.554 | 15 | Y | 3.5 | 28.87717386 |
| 69 | 976.3524541 | 0.050917677 | 2.706 | 14 | N | | 24.73444031 |
| 71 | 2585.986029 | 0.151257402 | 3.131 | 15 | Y | 13 | 25.68566847 |
| 72 | 135.575661 | 0.007750944 | 5.272 | 10 | Y | 8 | 15.63203413 |
| 73 | 2153.692923 | 0.117185844 | 2.716 | 21 | N | 7.5 | 18.96537664 |
| 74 | 265.5436466 | 0.012493827 | 9.313 | 16 | N | | 41.67792217 |

TABLE 1F

| subjectid | T_DISP_DIFF_PER | T_DIS_DIF | T_DIS_DIF_PER | T_AREA_DIF | T_AREA_DIF_PER |
|---|---|---|---|---|---|
| 1 | 0.164561192 | 49.92077556 | 0.175143422 | 3036.331734 | 0.296639207 |
| 2 | 0.411290142 | 85.04272977 | 0.197286745 | 9945.55049 | 0.357566578 |
| 3 | 0.036145823 | 0.648363253 | 0.001672596 | 41.66018306 | 0.001838401 |
| 4 | 0.055071506 | 21.80122377 | 0.061468573 | 3103.248389 | 0.210727177 |
| 5 | 0.006753958 | 4.364891045 | 0.010622905 | 694.8489554 | 0.026375966 |
| 7 | 0.044993681 | 5.199765166 | 0.013145364 | 894.1647825 | 0.036787579 |
| 8 | 0.041710798 | 27.31899879 | 0.07809499 | 2332.868003 | 0.121862872 |
| 9 | 0.005161389 | 5.326341023 | 0.011655783 | 387.4126839 | 0.013180696 |
| 10 | 0.016953318 | 6.691011566 | 0.011903199 | 1837.936772 | 0.037136726 |
| 11 | 0.027021184 | 3.623105843 | 0.00682645 | 1236.902101 | 0.028503655 |
| 12 | 0.049602486 | 30.79853416 | 0.072267953 | 3848.216705 | 0.138201067 |
| 13 | 0.042565303 | 19.41021588 | 0.051529996 | 2187.702409 | 0.113146581 |
| 14 | 1.37452E-05 | 6.811332329 | 0.018262049 | 863.6567378 | 0.041745042 |
| 15 | 2.16028E-05 | 0.442638359 | 0.000906571 | 526.6684836 | 0.014175078 |
| 16 | 0.047645591 | 21.19990226 | 0.063177717 | 2117.185083 | 0.126027397 |
| 17 | 0.031490491 | 31.72403759 | 0.072330376 | 3380.669343 | 0.115137748 |
| 18 | 0.029095578 | 45.6909944 | 0.075094658 | 7206.954404 | 0.129837241 |
| 19 | 0.124320176 | 83.9469159 | 0.143664125 | 12261.5201 | 0.232901265 |
| 21 | 0.025228973 | 57.77520587 | 0.108713665 | 8203.464636 | 0.19570638 |
| 22 | 0.013606843 | 25.99383533 | 0.053041959 | 3918.837943 | 0.105226642 |
| 23 | 0.051991608 | 1.291308064 | 0.003610729 | 254.1149226 | 0.013120301 |
| 24 | 0.107513947 | 39.59133982 | 0.101719382 | 3824.584585 | 0.187066217 |
| 25 | 0.084733071 | 46.18362433 | 0.089358297 | 5380.779759 | 0.139856484 |
| 26 | 0.065011496 | 24.54589772 | 0.060735514 | 2663.196328 | 0.112615805 |
| 27 | 0.079866848 | 38.88138361 | 0.106888396 | 2696.134172 | 0.159374334 |
| 29 | 0.064142617 | 5.622769265 | 0.013656516 | 1764.869608 | 0.068367508 |
| 30 | 3.42465E-06 | 1.083227404 | 0.003101123 | 127.3430461 | 0.006907823 |
| 31 | 0.017260558 | 9.785998 | 0.022296675 | 1372.971357 | 0.049014956 |
| 32 | 0.002831591 | 1.023161061 | 0.002689537 | 565.525223 | 0.026566143 |
| 33 | 0.002012454 | 11.55532142 | 0.027904647 | 1951.634429 | 0.075511555 |
| 34 | 0.021218634 | 6.942626647 | 0.016561841 | 444.6106247 | 0.016836161 |
| 35 | 0.00690477 | 11.64438161 | 0.028594143 | 1058.205074 | 0.042516939 |
| 36 | 0.04473257 | 0.028300163 | 7.85719E-05 | 198.4450306 | 0.009879934 |
| 37 | 0.017297442 | 43.67876548 | 0.104880899 | 4118.079592 | 0.16798479 |
| 38 | 0.026275687 | 8.780974206 | 0.023904258 | 531.3480541 | 0.0263172 |
| 39 | 0.441610334 | 45.41015349 | 0.106275058 | 5843.96675 | 0.254352657 |
| 40 | 0.014984938 | 12.5950685 | 0.028411838 | 2108.613771 | 0.08974212 |
| 41 | 0.042796627 | 17.19703354 | 0.043138987 | 2585.307497 | 0.108208105 |
| 42 | 0.058378389 | 40.69510376 | 0.098900861 | 4269.223828 | 0.167384369 |
| 43 | 0.041261609 | 22.9697763 | 0.056164536 | 1864.119107 | 0.078834951 |
| 44 | 0.208254575 | 17.80105191 | 0.038813794 | 2461.177187 | 0.075722366 |
| 45 | 0.003634754 | 11.11546327 | 0.02723661 | 894.749058 | 0.035508958 |
| 46 | 0.048692761 | 5.465899967 | 0.013663826 | 369.9707555 | 0.015382935 |
| 47 | 0.091407242 | 22.44113353 | 0.053126291 | 2818.001036 | 0.111936766 |
| 48 | 0.070220711 | 38.08179556 | 0.088987993 | 4522.138049 | 0.16735417 |
| 50 | 0.001293654 | 4.197367876 | 0.011690496 | 561.9289414 | 0.033169616 |
| 51 | 0.054144934 | 28.81204811 | 0.066130362 | 3502.488448 | 0.130510473 |
| 52 | 0.026978746 | 27.37299632 | 0.062349639 | 3323.726956 | 0.117156358 |
| 53 | 0.040424585 | 22.54594595 | 0.049459425 | 3465.494536 | 0.113152184 |
| 55 | 0.013215335 | 21.2949447 | 0.053144555 | 2816.051227 | 0.13999081 |
| 56 | 0.010362684 | 2.080875949 | 0.005680228 | 275.23167 | 0.015425066 |
| 57 | 0.071257889 | 14.01409696 | 0.033647757 | 1062.342846 | 0.042226251 |
| 59 | 0.178814567 | 44.6507168 | 0.122925289 | 3579.865588 | 0.189520097 |
| 61 | 0.050567951 | 5.424805882 | 0.013207461 | 205.6651598 | 0.008171698 |
| 62 | 0.180518038 | 19.04042463 | 0.043749383 | 1985.531764 | 0.082207635 |
| 63 | 0.012585661 | 1.813678108 | 0.005095672 | 447.3564409 | 0.024518656 |
| 65 | 0.007267696 | 7.69302894 | 0.016504414 | 291.8225855 | 0.00946913 |
| 66 | 0.015636634 | 6.877349069 | 0.017543329 | 444.7806252 | 0.01902992 |
| 67 | 0.089673725 | 10.53756072 | 0.027138219 | 1096.798805 | 0.048468658 |
| 68 | 0.090423607 | 24.82690167 | 0.058758859 | 2542.644836 | 0.097849735 |
| 69 | 0.093933298 | 19.03508405 | 0.051117472 | 2516.071616 | 0.116672692 |
| 71 | 0.091783908 | 15.99884713 | 0.040305982 | 2142.802059 | 0.08929055 |
| 72 | 0.050337241 | 2.918903521 | 0.006963416 | 185.2057471 | 0.007100506 |
| 73 | 0.053079143 | 18.54578645 | 0.04321612 | 2217.622459 | 0.092276301 |
| 74 | 0.115360652 | 23.528081 | 0.050917796 | 1985.195184 | 0.063121208 |

TABLE 1G

| subjectid | intervention_cat | intervention_binary |
|---|---|---|
| 1 | 2 | 0 |
| 2 | 4 | 1 |
| 3 | 3 | 1 |
| 4 | 2 | 0 |
| 5 | 3 | 1 |
| 7 | 3 | 1 |
| 8 | 2 | 0 |
| 9 | 3 | 1 |
| 10 | 2 | 0 |
| 11 | 4 | 1 |
| 12 | 3 | 1 |
| 13 | 3 | 1 |
| 14 | 3 | 1 |
| 15 | 3 | 1 |
| 16 | 3 | 1 |
| 17 | 2 | 0 |
| 18 | 3 | 1 |
| 19 | 4 | 1 |
| 21 | 4 | 1 |
| 22 | 3 | 1 |
| 23 | 4 | 1 |
| 24 | 4 | 1 |
| 25 | 3 | 1 |
| 26 | 4 | 1 |
| 27 | 3 | 1 |
| 29 | 3 | 1 |
| 30 | 3 | 1 |
| 31 | 4 | 1 |
| 32 | 2 | 0 |
| 33 | 1 | 0 |
| 34 | 2 | 0 |
| 35 | 4 | 1 |
| 36 | 3 | 1 |
| 37 | 4 | 1 |
| 38 | 3 | 1 |
| 39 | 4 | 1 |
| 40 | 4 | 1 |
| 41 | 4 | 1 |
| 42 | 3 | 1 |
| 43 | 3 | 1 |
| 44 | 4 | 1 |
| 45 | 3 | 1 |
| 46 | 2 | 0 |
| 47 | 3 | 1 |
| 48 | 4 | 1 |
| 50 | 2 | 0 |
| 51 | 3 | 1 |
| 52 | 3 | 1 |
| 53 | 3 | 1 |
| 55 | 3 | 1 |
| 56 | 3 | 1 |
| 57 | 3 | 1 |
| 59 | 3 | 1 |
| 61 | 3 | 1 |
| 62 | 2 | 0 |
| 63 | 2 | 0 |
| 65 | 1 | 0 |
| 66 | 3 | 1 |
| 67 | 2 | 0 |
| 68 | 3 | 1 |
| 69 | 3 | 1 |
| 71 | 2 | 0 |
| 72 | 3 | 1 |
| 73 | 2 | 0 |
| 74 | 1 | 0 |

TABLE 2

| | |
|---|---|
| subjectid | Unique identifier for Subject |
| new | Whether or Not Subject is New Patient |
| age | Age of Subject |
| gender | Gender of Subject |
| srs_available | Whether or Not SRS Scores are Available |
| srs_pain_hm | SRS Pain Score |
| srs_appearance_hm | SRS Appearance Score |
| srs_activity_hm | SRS Activity Score |
| srs_mental_hm | SRS Mental Score |
| srs_satisfaction_hm | SRS Satisfaction Score |
| srs_tscore_hm | SRS Total Score |
| srs_hmscore | SRS Heath Mindset Score |
| maxATR | Maximum Angle of Trunk Rotation |
| maxcobb | Maximum Cobb Score |
| maxsm | Maximum Scoliometer Measurement |
| TS_XR | Radiographic Trunk Shift |
| CB_XR | Radiographic Coronal Balance |
| CLAV_ANG_XR | Radiographic Clavicle Angle |
| TS_3D | 3D Scan Based Trunk Shift |
| CB_3D | 3D Scan Based Coronal Balance |
| CLAV_ANG_3D | 3D Scan Based Clavicle Angle |
| LATR | Lumbar Angle of Trunk Rotation |
| LACA | Lumbar Associated Cobb Angle |
| LSM_YN | Whether or Not a Lumber Spine |
| | Scoliometer Measurement is Available |
| LSM | Lumber Spine Scoliometer Measurement |
| L_DISP_DIFF | Lumbar Diff. Between Left and Right |
| | Displacements from Sagittal Line to |
| | Projection of Axis of Rotation on Horizon |
| L_DISP_DIFF_PER | Lumbar Diff. Between Left and |
| | Right Displacements from |
| | Sagittal Line to Projection of Axis of Rotation |
| | on Horizon as a % of Total Diameter |
| L_DIS_DIF | Lumbar Diff. Between Left and Right |
| | Circumferential Distance from Sagittal |
| | Line to Projection of Axis of Rotation on Horizon |
| L_DIS_DIF_PER | Lumbar Diff. Between Left and Right |
| | Circumferential Distance from Sagittal Line |
| | to Projection of Axis of Rotation on Horizon |
| | Line as a Percentage of Total Diameter |
| L_AREA_DIF | Lumbar Diff. Between Area of Left Upper |
| | Posterior Quadrant and Right |
| | Upper Posterior Quadrant |
| L_AREA_DIF_PER | Lumbar Diff. Between Area of Left Upper |
| | Posterior Quadrant and Right Upper Posterior |
| | Quadrant as a Percentage of Total Area |
| | in Posterior Hemisphere |
| TATR | Thoracic Angle of Trunk Rotation |
| TACA | Thoracic Associated Cobb Angle |
| TSM_YN | Whether or Not a Thoracic Spine Scoliometer |
| | Measurement is Available |
| TSM | Thoracic Spine Scoliometer Measurement |
| T_DISP_DIFF | Thoracic Diff. Between Left and Right |
| | Displacements from Sagittal Line to |
| | Projection of Axis of Rotation on Horizon |
| T_DISP_DIFF_PER | Thoracic Diff. Between Left and Right |
| | Displacements from Sagittal Line to Projection |
| | of Axis of Rotation on Horizon as a % of |
| | Total Diameter |
| T_DIS_DIF | Thoracic Diff. Between Left and Right |
| | Circumferential Distance from Sagittal Line |
| | to Projection of Axis of Rotation on Horizon |
| T_DIS_DIF_PER | Thoracic Diff. Between Left and Right |
| | Circumferential Distance from Sagittal Line |
| | to Projection of Axis of Rotation on Horizon |
| | as a % of Total Diameter |
| T_AREA_DIF | Thoracic Diff. Between Area of |
| | Left Upper Posterior Quadrant |
| | and Right Upper Posterior Quadrant |
| T_AREA_DIF_PER | Thoracic Diff. Between Area of Left Upper |
| | Posterior Quadrant and Right Upper Posterior |
| | Quadrant as a % of Total Area in |
| | Posterior Hemisphere |
| intervention_cat | 0 = No Intervention Necessary; 1 = Follow-Up, |
| | But No Intervention; 2 = Bracing; 3 = Surgery |
| intervention_binary | 1 = Any Intervention Necessary (Surgery |
| | or Bracing); 0 = No Intervention Necessary |

What is claimed is:

1. A three dimensional diagnostic system comprising:
a three dimensional scanning device capable of obtaining a three dimensional scan of a human body without emitting ionizing or other damaging radiation; and
a computing device in communication with the three dimensional scanning device and capable of generating a mesh from a three dimensional scan and analyzing said mesh to identify a musculoskeletal anomaly.

2. The system of claim 1, wherein the three dimensional scanning device is a white light scanning camera or a LiDAR-enabled camera.

3. The system of claim 1, wherein the computing device is a mobile device.

4. The system of claim 3, wherein the mobile device is selected from a mobile phone, a tablet, a laptop computer, or a notebook computer.

5. The system of claim 1, wherein the computing device is capable of transmitting data over a network.

6. The system of claim 1, further comprising a remote server connected to the computing device via a network. 7 A method for detecting and monitoring scoliosis comprising:
obtaining a 3D topographic scan of a subject's body using a 3D topographic imaging device;
analyzing the 3D topographic scan by:
identifying a plurality of key feature points on the regions of the 3D topographic scan reflecting the back of the subject;
measuring a distance or angle between at least a first key feature point and a second key feature point in the plurality of key feature points;
identifying scoliosis based on the distances, angles, and volumetric relationships quantified in upright and bending poses;
classifying the scoliosis as in need of orthopaedic referral or not in need of orthopaedic referral; and
classifying the scoliosis as operative, eligible for casting and/or bracing or not in need of intervention; and
treating the subject based on the classification of the scoliosis.

8. The method of claim 7, wherein the plurality of key features are selected from the group consisting of: the midsternal notch, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

9. The method of claim 7, wherein the treating step includes a surgical operation, other non-surgical intervention, or physical therapy.

10. The method of claim 9, further comprising:
obtaining a second 3D topographic scan of the subject's body post-treatment;
identifying a second plurality of key feature points in the second 3D topographic scan using a fracture detector;
measuring a distance, angle, or volumetric change between at least a first key feature point and a second key feature point in the second plurality of key feature points using the fracture detector;
calculating the difference in the measured distance, angles or volumetric change; and
tracking the subject's recovery based on the calculated differences in distances, angles or volumetric measurements of interest.

11. The method of claim 10, wherein the plurality of key features are selected from the group consisting of: the midsternal notch, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

12. The method of claim 7, wherein the 3D topographic scan is accomplished using a structured light scanner or LiDAR-enabled camera.

13. The method of claim 12, wherein the 3D topographic scan is accomplished using a mobile device.

14. The method of claim 13, wherein the mobile device is selected from a mobile phone or tablet.

15. A method for detecting and treating clavicle fractures comprising:
obtaining a 3D topographic scan of a subject's body using a 3D topographic imaging device;
identifying a plurality of key feature points on the regions of the 3D topographic scan reflecting the shoulders and back of the subject;
measuring a distance between at least a first key feature point and a second key feature point in the plurality of key feature points;
identifying a clavicle fracture based on the distance;
classifying the clavicle fracture as operative or non-operative; and
treating the subject based on the classification of the clavicle fracture.

16. The method of claim 15, wherein the plurality of key features are selected from the group consisting of: the midsternal notch, the acromial process, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

17. The method of claim 15, wherein the treating step includes a surgical operation.

18. The method of claim 17, further comprising:
obtaining a second 3D topographic scan of the subject's body post-operatively;
identifying a second plurality of key feature points in the second 3D topographic scan using a fracture detector;
measuring a distance between at least a first key feature point and a second key feature point in the second plurality of key feature points using the fracture detector;
calculating the difference in the measured distances;
calculating volumetric relationships within 3D scans; and
tracking the subject's recovery based on the calculated differences.

19. The method of claim 18, wherein the plurality of key features are selected from the group consisting of: the midsternal notch, the superior/anterior aspect of the acromioclavicular joint, the posterior/lateral border of the acromion, the C7 spinous process, the inferior angle of the scapula, the nipples, the olecranon process, the anterior superior iliac spine, greater trochanter, patella, ankle malleoli, and digits.

20. The method of claim 15, wherein the 3D topographic scan is accomplished using a structured light scanner or LiDAR-enabled camera.

21. The method of claim 20, wherein the 3D topographic scan is accomplished using a mobile device.

22. The method of claim 21, wherein the mobile device is selected from a mobile phone or tablet.

23. A method for detecting musculoskeletal anomalies comprising:
obtaining a 3D topographic scan of a subject's body using a 3D topographic imaging device;
performing range of motion, center of gravity, asymmetry, or posture analysis on the 3D topographic scan by bisecting the scan with one or more lines and measuring a key feature along the one or more lines; and
identifying a musculoskeletal anomaly based on the distance.

24. The method of claim 23, wherein the 3D topographic scan is accomplished using a structured light scanner or LiDAR-enabled camera.

25. The method of claim 23, wherein the 3D topographic scan is accomplished using a mobile device.

26. The method of claim 25, wherein the mobile device is selected from a mobile phone or tablet.

27. The method of claim 23, wherein the musculoskeletal anomaly is selected from scoliosis, back pain, neck pain, joint pain, sarcopenia, arthritis, osteoporosis, bone and soft tissue injury.

28. The method of claim 23, wherein obtaining the 3D topographic scan is accomplished by converting one or more two-dimensional images into a 3D representation of the subject's body.
